SEQUENCE LISTING (20 sequences)

SEQ ID NO: 1 - protein, 10 aa, Homo sapiens
  REGION 1..10: note = Description of sequence: non-canonical peptide, associated gene: RSPRY1, classification: 5'-UTR, short form name: p1
ATFFGIQLLK

SEQ ID NO: 2 - protein, 11 aa, Homo sapiens
  REGION 1..11: note = Description of sequence: non-canonical peptide, associated gene: SERPINB6, classification: 5'-UTR, short form name: p2
AVYQEPALFWK

SEQ ID NO: 3 - protein, 9 aa, Homo sapiens
  REGION 1..9: note = Description of sequence: non-canonical peptide, associated gene: ZNF32, classification: 5'-UTR, short form name: p3
EVVTEVVSR

SEQ ID NO: 4 - protein, 11 aa, Homo sapiens
  REGION 1..11: note = Description of sequence: non-canonical peptide, associated gene: CDK16, classification: 5'-UTR, short form name: p4
MAEDYWDGRLR

SEQ ID NO: 5 - protein, 9 aa, Homo sapiens
  REGION 1..9: note = Description of sequence: non-canonical peptide, associated gene: NUDT1, classification: 5'-UTR, short form name: p5
MYWSNQITR

SEQ ID NO: 6 - protein, 9 aa, Homo sapiens
  REGION 1..9: note = Description of sequence: non-canonical peptide, associated gene: TMEM68, classification: CDS (frame-shift), short form name: p6
NTFNTSLLY

SEQ ID NO: 7 - protein, 9 aa, Homo sapiens
  REGION 1..9: note = Description of sequence: non-canonical peptide, associated gene: CIAO2A, classification: CDS (frame-shift), short form name: p7
QSPVALRPL

SEQ ID NO: 8 - protein, 9 aa, Homo sapiens
  REGION 1..9: note = Description of sequence: non-canonical peptide, associated gene: LINC02085, classification: lncRNA, short form name: p8
SLLIDGPQV

SEQ ID NO: 9 - protein, 11 aa, Homo sapiens
  REGION 1..11: note = Description of sequence: non-canonical peptide, associated gene: CRYBG1, classification: CDS (frame-shift), short form name: p9
STIHLASQFTK

SEQ ID NO: 10 - protein, 9 aa, Homo sapiens
  REGION 1..9: note = Description of sequence: non-canonical peptide, associated gene: PLOD3, classification: 5'-UTR, short form name: p10
SVAPASGAF

SEQ ID NO: 11 - protein, 9 aa, Homo sapiens
  REGION 1..9: note = Description of sequence: non-canonical peptide, associated gene: DNAJC22, classification: 5'-UTR, short form name: p11
TEDPETSVL

SEQ ID NO: 12 - protein, 11 aa, Homo sapiens
  REGION 1..11: note = Description of sequence: non-canonical peptide, associated gene: PPP1R14B-AS1, classification: lncRNA, short form name: p12
VLPDQHLVTTV

SEQ ID NO: 13-18 - skipped (6 entries)

SEQ ID NO: 19 - protein, 9 aa, synthetic construct
  REGION 1..9: note = Description of sequence: Postive Control
MCDEDETTA

SEQ ID NO: 20 - protein, 9 aa, synthetic construct
  REGION 1..9: note = Description of sequence: Negative Control
SLLMWITQV

(19) United States
(12) Patent Application Publication
(10) Pub. No.: US 2026/0028382 A1
(43) Pub. Date: Jan. 29, 2026

(54) IMMUNOGENIC PEPTIDES AND THEIR USES IN CANCER DIAGNOSIS AND THERAPY

(71) Applicants: The Chinese University of Hong Kong, Shatin (CN); The University of Hong Kong

(72) Inventors: Nathalie WONG, New Territories (CN); Wing Hon Jason WONG, Hong Kong (CN); Yujuan DONG, Sha Tin (CN); Pak Shun LI, Hong Kong (CN)

(21) Appl. No.: 19/218,053

(22) Filed: May 23, 2025

Related U.S. Application Data
(60) Provisional application No. 63/652,519, filed on May 28, 2024.

(51) Int. Cl. C07K 14/47; A61K 39/00; G01N 33/68

(52) U.S. Cl. CPC C07K 14/4748; A61K 39/0011; G01N 33/6848; A61K 2039/54

(57) ABSTRACT

The present invention resides in the discovery of novel immunogenic peptides that are apparently encoded by RNA previously thought as non-coding in nature. The presence of these peptides is associated with occurrence and progression of malignancies such as hepatocellular carcinoma (HCC). Thus, the diagnostic and therapeutic applications of these peptides are also disclosed.

### RELATED APPLICATIONS

This application claims priority to U.S. Provisional Patent Application No. 63/652,519, filed May 28, 2024, the contents of which are hereby incorporated by reference in the entirety for all purposes.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Sep. 22, 2025, is entitled “080015-1497249-041210US_ST26.xml”, and is 20,468 bytes in size.

### BACKGROUND OF THE INVENTION

Cancer-related causes are among the top reasons of death in developed countries. In the US alone, the number of new cases of cancer of any site averages about 450 per 100,000 men and women per year, and the number of deaths averages about 170 per 100,000 men and women per year. Cancer is a disease with a high mortality rate: while about 1,700,000 newly diagnosed cancer cases are expected each year, over 600,000 deaths annually are attributable to various types of cancer. Based on data from recent years, it is estimated that over 38% of men and women will be diagnosed with cancer at some point during their lifetime.

Given the prevalence of cancer and its significant health implications, there exists an urgent need for developing new compositions and methods useful for the early diagnosis and effective treatment of the disease. This invention provides information to fulfill these and other related needs.

### BRIEF SUMMARY OF THE INVENTION

The present inventors discovered for the first time several immunogenic peptides that are associated with malignancies such as hepatocellular carcinoma (HCC). Thus, in a first aspect, the present invention provides an isolated peptide consisting of the amino acid sequence set forth in any one of SEQ ID NOs: 1-12 plus one or more heterologous amino acid sequence optionally present in the peptide, which may be located at the C-terminus or N-terminus or both termini of the peptide. In some embodiments, the peptide of the present invention is no longer than 15, 20, 25, 30, 50, 80, 100, 150, 200, 250, or 300 amino acids in total length. In some embodiments, the peptide consists of the amino acid sequence set forth in SEQ ID NO: 1, 2, 3, 4, 5, 6, 7, 8, 9, 10, 11, or 12. In some embodiments, the peptide of the present invention as described above and herein is a part of a conjugate, which includes the peptide and a heterologous moiety conjugated to the peptide as its conjugation partner. In some embodiments, the heterologous moiety is a detectable moiety that is capable of emitting a detectable signal. In some embodiments, the heterologous moiety is a solid substrate.

In a second aspect, the present invention provides a nucleic acid comprising a polynucleotide sequence, which encodes the peptide of this invention as described above and herein. In some embodiments, the nucleic acid is a DNA molecule. In some embodiments, the nucleic acid is an RNA molecule. Optionally, the nucleic acid may include one or more artificial or non-nature nucleotides. In some embodiments, the nucleic acid is in form of an expression cassette, which comprises a heterologous promoter sequence operably linked to the polynucleotide coding sequence and driving the transcription of the coding sequence. In some embodiments, the expression cassette is a part of a vector, such as a bacterial plasmid or a viral vector (e.g., a full or partial viral genome).

In a related aspect, the present invention provides a composition comprising (1) the peptide of the present invention as described above or herein or the nucleic acid encoding the peptide; and (2) one or more pharmaceutically acceptable excipient. In some embodiments, the composition is formulated for delivery to a recipient by injection. In some embodiments, the composition comprises an adjuvant to enhance an immune response the composition is intended to induce in the recipient.

The third aspect of the present invention provides a method for detecting presence of cancerous cells in a patient. The method comprises a step of detecting, in a biological sample obtained from a patient who is being tested for a possible cancer condition, the presence of at least one, preferably more (e.g., two, three, or more) of the peptides described above and herein. For example, the presence of at least one, two, three, or more of the peptides P1-P12, especially P2, P3, and P10 and possibly more, is sought to be ascertained. In some embodiments, the biological sample is a blood or tissue sample comprising lymphocytes. For example, a peripheral blood mononucleated cell (PBMC) sample may be used in testing. In some embodiments, detecting of the peptides is focused on the MHC complex present on the lymphocytes. In some embodiments, detecting of the peptides involves the use of mass spectrometry. In some embodiments, detecting the presence of at least three peptides, preferably at least the peptides having the amino acid sequences set forth in SEQ ID NOs: 2, 3, and 10, leads to a determination of cancer presence in the patient. In some embodiments, the cancer is liver cancer, such as hepatocellular carcinoma (HCC).

In a fourth aspect, the present invention provides a method for treating cancer in a patient who has been diagnosed with cancer or for reducing cancer risk in a patient who is deemed to be at an increased risk of later developing cancer. The method includes the step of administering to a patient in need thereof a composition comprising an effective amount of the peptide of this invention as described above herein or an effective amount of a nucleic acid encoding the peptide. In some embodiments, the method is practiced on a patient who is at risk of developing liver cancer or has received a diagnosis of liver cancer. In some embodiments, the liver cancer is hepatocellular carcinoma (HCC). In some embodiments, the administering step comprises injecting the composition to the patient.

In a further aspect of this invention, a kit is provided for treating cancer by inhibiting cancer cell proliferation. Typically, the kit includes a plurality of containers, with a first container containing a composition comprising an effective amount of the peptide of this invention as described above herein or an effective amount of a nucleic acid encoding the peptide, and a second container containing a known anti-cancer therapeutic agent. In some embodiments, the composition comprises the peptides having the amino acid sequences set forth in SEQ ID NOs: 2, 3, and 10. In some embodiments, the kit includes multiple containers each containing a composition that includes one different peptide comprising a distinct amino acid sequence as set forth in SEQ ID NOs: 1-12. In some embodiments, the cancer being treated is liver cancer, especially hepatocellular carcinoma (HCC). In some embodiments, the kit may further comprise user instructions.

### BRIEF DESCRIPTION OF THE DRAWINGS

FIG. 1A-FIG. 1E: HLA-A/B/C immunoaffinity purification and antigen enrichment. FIG. 1A. Graphical summary of HLA-A/B/C immunoaffinity purification and antigen enrichment. Three quality control steps (FIG. 1c-e) were indicated in the Workflow. Image was created using BioRender software. FIG.-1B. HCC cell line surface HLA-class I molecules expression was determined with immunostaining followed by FACS analysis. FIG. 1C. The input and flow-through of IgG or HLA-A/B/C antibody after binding with protein A beads was evaluated by western blot analysis. Equal amount of IgG and HLA-A/B/C Ab were used in the immunoaffinity columns. FIG. 1D. The HLA complex in the cell lysis before and after binding with immunoaffinity column was determined by western blot analysis. FIG. 1E. HLA-A/B/C molecules eluted by 80% ACN was detected by western blot analysis. Representative images were shown.

FIG. 2A-FIG. 2B: Overview of immunopeptidome data generated from HCC patients. FIG. 2A. Distribution of peptide lengths of the generated immunopeptidome. FIG. 2B. Correlation between calibrated retention time and predicted hydrophobicity.

FIG. 3: Representative spectra matching to the identified non-canonical peptides. All figures were generated from the PDV software.

FIG. 4A-FIG. 4H: Immunogenic potential of the HCC patient-derived cell lines HLA-peptidome. FIG. 4A. Identification of Patient-Specific HLA Alleles: All possible patient-specific HLA alleles were identified using LinkSeq HLA typing, represented at the 4-digit resolution. FIG. 4B-FIG. 4C. NetMHCPan Prediction for Each Patient: Each identified peptide was queried against all possible patient-specific HLA alleles. Separate plots were shown for ranking by EL and BA datasets. FIG. 4D. The consensus motif of HKCI-4 HLA-peptidome (SEQ ID NO: 13) FIG. 4E. Consensus motif of HLA-A0201 according to MHC Motif Viewers (SEQ ID NO: 14). FIG. 4F. The consensus motif of HKCI-C1 HLA-peptidome (SEQ ID NO: 15) FIG. 4G. Consensus motif of HLA-A2402 according to MHC Motif Viewers (SEQ ID NO: 16). FIG. 4H. The consensus motif of HKCI02 HLA-peptidome (SEQ ID NO: 19) FIG. 4I. Consensus motif of HLA-A1101 according to MHC Motif Viewers. Only MHC Motif Viewers from the EL training dataset was shown here, as the consensus motif derived from EL and BA training dataset was mostly similar to each other (SEQ ID NO: 18).

FIG. 5A-FIG. 5I: Expression and visualisation of the twelve non-canonical novel peptides. FIG. 5A. Expression heatmap of the candidates peptides, either as TPM value for Illumina data, or as binary presence heatmap for peptide and long-read data FIG. 5B. Read length distribution of the candidate peptides. FIG. 5C. Classification based on genomic positions of the candidate peptides. FIG. 5D-FIG. 5F. IGV visualisation of representative peptides and ORFs derived from 5′-UTR. FIG. 5G-FIG. 5H. IGV imagas of lncRNA-derived peptides and ORFs FIG. 5I. IGV image of p9. an example of peptides produced from frame-shift ORF of a canonical exon

FIG. 6A-FIG. 6C: Validation of the immunogenicity of candidate peptides using IFN-γ ELISpot assay. FIG. 6A. Peptides used in the ELISpot assay (SEQ ID NOs: 19, 20, 2, 9 and 3 respectively top to bottom). FIG. 6B. Images of the ELISpot assay showed the IFN-γ secretion by DC:CD8+ T cells co-culture following 24 hours stimulation with the anti-CD3 antibody or various peptides. Autologous DC and CD8+ T cells isolated from healthy donors were used in the analysis. FIG. 6C. Summary of the IFN-γ spot number generated in FIG. 6B. Statistical analysis was performed against Actin1-9 peptide using unpaired t-test. Data are presented as mean values±SD. *p-value<0.05, **p-value<0.01 ***p-value<0.001, ****p-value<0.0001.

FIG. 7A-FIG. 7G: Immunogenicity and tumor-specificity assessment of the twelve key peptides. FIG. 7A-FIG. 7D. NetMHCPan prediction results for the interactions between the twelve key peptides and the HLA alleles of the patient-derived cell line used in this study. Pie chart area represents the number of HLA interactions predicted as strong or weak binding interactions, based on ranking percentile of eluted ligands (EL) and binding affinity (BA) training dataset. FIG. 7E-FIG. 7F. Patient-specific peptide immunogenicity based on predictions from EL and BA training dataset. Heatmap color key shows ranking percentile of strongest interaction between a peptide and all possible alleles of each patient. IEatlas data for the peptides were included as the row annotation, indicating detection in published mass spectrometry data, tumor specificity, and homology to foreign antigen according to IEDB. FIG. 7G. DeepImmuno evaluation of peptide-HLA interactions. including strong and weak binders from either EL or BA. HLA alleles with the same 2-digits but different 4-digits HLA types were grouped together. Results sorted by highest to lowest DeepImmuno scores. The 10 peptide-HLA interactions with highest scores listed on right side of figure.

### DEFINITIONS

The term “nucleic acid” or “polynucleotide” refers to deoxyribonucleotides or ribonucleotides and polymers thereof in either single-or double-stranded form. Unless 30) specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); and Cassol et al., (1992); Rossolini et al., Mol. Cell. Probes, 8:91-98 (1994)). The terms nucleic acid and polynucleotide are used interchangeably with gene, cDNA, and mRNA encoded by a gene.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An “antibody” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂. a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology.

Further modification of antibodies by recombinant technologies is also well known in the art. For instance, chimeric antibodies combine the antigen binding regions (variable regions) of an antibody from one animal with the constant regions of an antibody from another animal. Generally, the antigen binding regions are derived from a non-human animal, while the constant regions are drawn from human antibodies. The presence of the human constant regions reduces the likelihood that the antibody will be rejected as foreign by a human recipient. On the other hand, “humanized” antibodies combine an even smaller portion of the non-human antibody with human components. Generally, a humanized antibody comprises the hypervariable regions, or complementarity determining regions (CDR), of a non-human antibody grafted onto the appropriate framework regions of a human antibody. Antigen binding sites may be wild type or modified by one or more amino acid substitutions, e.g., modified to resemble human immunoglobulin more closely. Both chimeric and humanized antibodies are made using recombinant techniques, which are well-known in the art (see, e.g., Jones et al. (1986) Nature 321:522-525).

Thus, the term “antibody,” as used herein, also includes antibody fragments either produced by the modification of whole antibodies or antibodies synthesized de novo using recombinant DNA methodologies (e.g., single chain Fv, a chimeric or humanized antibody). One example is the so-called “nanobody” or single-domain antibody (sdAb), an antibody fragment consisting of a single monomeric variable antibody domain, especially a heavy chain variable domain. Like a whole antibody, a nanobody is able to bind selectively to a specific antigen. With a molecular weight of only 12-15 kDa, nanobodies are much smaller than common antibodies (150-160 kDa) having two heavy chains and two light chains, and even smaller than Fab fragments (˜50 kDa) and single-chain variable fragments (˜25 kDa).

The phrase “specifically binds,” when used in the context of describing a binding relationship of a particular molecule to a protein or peptide, refers to a binding reaction that is determinative of the presence of the protein in a heterogeneous population of proteins and other biologics. Thus, under designated binding assay conditions, the specified binding agent (e.g., an antibody) binds to a particular protein at least two times the background and does not substantially bind in a significant amount to other proteins present in the sample. Specific binding of an antibody under such conditions may require an antibody that is selected for its specificity for a particular protein or a protein but not its similar “sister” proteins. A variety of immunoassay formats may be used to select antibodies specifically immunoreactive with a particular protein or in a particular form. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective binding reaction will be at least twice background signal or noise and more typically more than 10, 20, 50, or up to 100 times background. On the other hand, the term “specifically bind” when used in the context of referring to a polynucleotide sequence forming a double-stranded complex with another polynucleotide sequence describes “polynucleotide hybridization” based on the Watson-Crick base-pairing, as provided in the definition for the term “polynucleotide hybridization method.”

As used herein, the term “cancer” encompasses various malignant neoplasms characterized by the proliferation of anaplastic cells that tend to invade surrounding tissue and metastasize to new body sites. Non-limiting examples of different types of cancer suitable for treatment using the compositions and methods of the present invention include colorectal cancer, colon cancer, anal cancer, liver cancer, ovarian cancer, breast cancer, lung cancer, bladder cancer, thyroid cancer, pleural cancer, pancreatic cancer, cervical cancer, prostate cancer, testicular cancer, bile duct cancer, gastrointestinal carcinoid tumors, esophageal cancer, gall bladder cancer, rectal cancer, appendix cancer, small intestine cancer, stomach (gastric) cancer, renal cancer (e.g., renal cell carcinoma), cancer of the central nervous system, skin cancer, oral squamous cell carcinoma, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma.

An “expression cassette” is a nucleic acid construct, generated recombinantly or synthetically, with a series of specified nucleic acid elements that permit transcription of a particular polynucleotide sequence (e.g., one encoding a polypeptide sequence comprising or consisting of a cancer-associated immunogenic peptide of this invention) in a host cell. An expression cassette may be part of a plasmid, viral genome, or nucleic acid fragment. Typically, an expression cassette includes a polynucleotide to be transcribed, operably linked to a promoter. “Operably linked” in this context means two or more genetic elements, such as a polynucleotide coding sequence and a promoter, placed in relative positions that permit the proper biological functioning of the elements, such as the promoter directing transcription of the coding sequence. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

The term “heterologous,” as used in the context of describing the relative location of two elements, refers to the two elements such as two polynucleotide sequences (e.g., a promoter and a polypeptide-encoding sequence) or polypeptide sequences (e.g., a first amino acid sequence (such as one set forth in SEQ ID NO: 1, 2, 3, 4, 5, 6, 7, 8, 9, 10, 11, or 12) and a second peptide sequence serving as a fusion partner with the first amino acid sequence) that are not naturally found in the same relative position. Thus, a “heterologous promoter” of a gene refers to a promoter that is not naturally operably linked to that gene. Similarly, a “heterologous polypeptide/amino acid sequence” or “heterologous polynucleotide” to a first amino acid sequence or its encoding sequence is one derived from an origin different from the first amino acid sequence or its encoding sequence or derived from the same origin but not naturally connected to the first amino acid sequence or its encoding sequence in the same fashion. The fusion of a cancer-associate immunogenic peptide of this invention (or its coding sequence) with a heterologous polypeptide (or polynucleotide sequence) does not result in a longer polypeptide or polynucleotide sequence that can be found in nature.

As used in this application, an “increase” or a “decrease” refers to a detectable positive or negative change in quantity from a comparison control, e.g., an established standard control (such as an average rate of cancer cell proliferation). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher.” and “lower,” as well as terms indicating an action to cause such changes or differences, such as “increase,” “promote.” “enhance,” “decrease.” “inhibit,” and “suppress,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

A composition “consisting essentially of an immunogenic peptide” is one that includes a cancer-associated immunogenic peptide of this invention and is able to inhibit or suppress specific symptoms related to cancer (such as HCC) but no other compounds that affect, either positively or negatively, to a detectable extent to the same inhibitive or suppressive activity. Such compounds may include one or more inactive excipients, e.g., for formulation or stability of a pharmaceutical composition, or other unrelated active ingredients that do not significantly alter the cancer inhibition or suppression activity in any measurable manner. Exemplary compositions consisting essentially of an immunogenic peptide include therapeutics, medicaments, and pharmaceutical compositions.

As used herein, an “effective amount” or a “therapeutically effective amount” means the amount of a compound that, when administered to a subject or patient for treating a disorder, is sufficient to prevent, reduce the frequency of, or alleviate the symptoms of the disorder. The effective amount will vary depending on a variety of the factors, such as a particular compound used, the disease and its severity, the age, weight, and other factors of the subject to be treated. Amelioration of a symptom of a particular condition by administration of a pharmaceutical composition described herein refers to any lessening. whether permanent or temporary, that can be associated with the administration of the pharmaceutical composition. For example, the amount of an immunogenic peptide of this invention is considered therapeutically effective for treating a condition involving undesired cellular proliferation when treatment results in eliminated symptoms, delayed onset of symptoms, or reduced frequency or severity of symptoms such as tumor growth, metastasis, etc.

As used herein, the term “treatment” or “treating” includes both therapeutic and preventative measures taken to address the presence of a disease or condition or the risk of developing such disease or condition at a later time. It encompasses therapeutic or preventive measures for alleviating ongoing symptoms, inhibiting or slowing disease progression, delaying of onset of symptoms, or eliminating or reducing side-effects caused by such disease or condition. A preventive measure in this context and its variations do not require 100% elimination of the occurrence of an event; rather, they refer to a suppression or reduction in the likelihood or severity of such occurrence or a delay in such occurrence.

A “subject,” or “subject in need of treatment.” as used herein, refers to an individual who seeks medical attention due to risk of, or actual sufferance from, a condition involving an undesirable or uncontrolled cellular proliferation resulting in benign or malignant tumorigenesis such as HCC. The term subject can include both animals, especially mammals, and humans. Subjects or individuals in need of treatment include those that demonstrate symptoms of neoplasm or are at risk of later developing these conditions and/or related symptoms.

The term “about” when used in reference to a given value denotes a range encompassing±10% of the value: for example, “about 10” denotes a range of 10±1 or 9-11.

A “pharmaceutically acceptable” or “pharmacologically acceptable” excipient is a substance that is not biologically harmful or otherwise undesirable, i.e., the excipient may be administered to an individual along with a bioactive agent without causing any undesirable biological effects to a detectable extent. Neither would the excipient interact in a deleterious manner with any of the components of the composition in which it is contained.

The term “excipient” refers to any essentially accessory substance that may be present in the finished dosage form of the composition of this invention. For example, the term “excipient” includes vehicles, binders, disintegrants, fillers (diluents), lubricants, glidants (flow enhancers), compression aids, colors, sweeteners, preservatives, suspending/dispersing agents, film formers/coatings, flavors and printing inks.

A “label,” “detectable label,” or “detectable moiety” is a composition detectable by radiological, spectroscopic, photochemical, biochemical, immunochemical, chemical, or other physical means. For example, useful labels include radioisotopes such as ³²P, fluorescent dyes, electron-dense reagents, enzymes (e.g., as commonly used in an ELISA), biotin, digoxigenin, or haptens and proteins that can be made detectable, e.g., by incorporating a radioactive component into a polypeptide or used to detect antibodies specifically reactive with the polypeptide. Typically a detectable label is a heterologous moiety attached to a probe or a molecule (e.g., a peptide or nucleic acid) with defined binding characteristics (e.g., a polypeptide or a polynucleotide with a known binding specificity), so as to allow the presence of the probe/molecule (and therefore its binding target) to be readily detectable. The heterologous nature of the label ensures that it has an origin different from that of the probe or molecule that it labels, such that the probe/molecule attached with the detectable label does not constitute a naturally occurring composition (e.g., a naturally occurring polynucleotide or polypeptide sequence).

### DETAILED DESCRIPTION OF THE INVENTION

### I. Introduction

The present invention relates to novel cancer-associated, immunogenic peptides, an immunostimulatory composition comprising at least one of such neoantigen peptides that can elicit an adaptive immune response for the treatment of a substantial proportion of subjects suffering from cancer, or can provide a new basis for cancer diagnosis, or can serve as a platform for the further development of pharmaceutically/immunologically active compounds and cells.

Immune checkpoint blockades (ICB) of immunosuppressive surface proteins have modest efficacy as a monotherapy in hepatocellular carcinoma (HCC). Cancer vaccine may enhance responses to ICB through the induction of tumor-specific immunity. The present inventors identified 12 neoantigens (P1-P12, corresponding to the amino acid sequences of SEQ ID NOs: 1-12, respectively) that are highly specific to HCC and can be used as markers for HCC diagnosis or as cancer vaccine for HCC. As such, this invention provides a vaccine composition comprising at least one of the 12 neoantigen peptides or a nucleic acid encoding at least one such peptides, which can be a useful tool for improving tumor immunotherapy and minimizing HCC progression.

### II. Recombinant Expression of Polypeptides

### A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange HPLC as described in Pearson & Reanier, J. Chrom. 255:137-149 (1983).

The sequence of a nucleic acid of interest, e.g., a polynucleotide sequence encoding a polypeptide having the amino acid sequence of any one of SEQ ID NOs: 1-12, and synthetic oligonucleotides can be verified after cloning or subcloning using, e.g., the chain termination method for sequencing double-stranded templates of Wallace et al., Gene 16:21-26 (1981).

The polypeptides being synthesized or recombinantly produced in accordance with the present invention may comprise or consist of one or the newly identified immunogenic peptides P1-P12, corresponding to the amino acid sequences set forth in SEQ ID NOs: 1-12. In some embodiments, such polypeptides are small peptides the same as or similar to the size of P1-P12, for example, in the length range of 9-11 (e.g., about 9, 10, or 11) amino acids or 10-15, 10-20, 10-25, 10-30, 10-40 or 10-50 amino acids, or up to about 60, 70, 80, 90, or 100 amino acids. The peptide of this invention in some cases has exactly the same amino acid sequence of any one of SEQ ID NOs: 1-12, ranging from 9, 10, to 11 amino acids in length. In other cases, it may further include one or more heterologous amino acid sequences, in addition to the peptide of any one of PI-P12, the heterologous amino acid sequence(s) derived from a source other than that of the P1-P12 peptide, respectively. Such heterologous amino acid sequence(s) may be located at the N-terminus and/or C-terminus of the 20) recombinant polypeptide. For example, a polypeptide comprising one of the P1-P12 peptides may optionally further include one or more additional heterologous amino acid sequence(s) of 1, 2, 3, 4, 5, 6, 7, 8, 9, 10, 11, 12, 13, 14, 15, 16, 17, 18, 19, 20, 25, 30, 35, 40, 45, or up to 50 amino acids at C- and/or N-terminus of the polypeptide sequence. Such heterologous peptide sequences can be of a varying nature, for example, any one of the “tags” known and used in the field of recombinant proteins: a peptide tag such as an AviTag, a peptide allowing biotinylation by the enzyme BirA and so the protein can be isolated by streptavidin, a Calmodulin-tag, a peptide bound by the protein calmodulin, a polyglutamate tag, a peptide binding efficiently to anion-exchange resin such as Mono-Q, an E-tag, a peptide recognized by an antibody, a FLAG-tag, a peptide recognized by an antibody, an HA-tag, a peptide recognized by an antibody, a His-tag, 5-10 histidines bound by a nickel or cobalt chelate, a Myc-tag, a short peptide recognized by an antibody, an S-tag, an SBP-tag, a peptide that specifically binds to streptavidin, a Softag 1 for mammalian expression, a Softag 3 for prokaryotic expression, a Strep-tag, a peptide that binds to streptavidin or the modified streptavidin called streptactin (Strep-tag II), a TC tag, a tetracysteine tag that is recognized by FLASH and ReAsH biarsenical compounds, a V5 tag, a peptide recognized by an antibody, a VSV-tag, a peptide recognized by an antibody, an Xpress tag; or a covalent peptide tags such as an Isopeptag, a peptide that binds covalently to pilin-C protein, a Spy Tag, a peptide that binds covalently to Spy Catcher protein; or a protein tag such as a BCCP tag (Biotin Carboxyl Carrier Protein), a protein domain biotinylated by BirA enabling recognition by streptavidin, a Glutathione-S-transferase (GST) tag, a protein that binds to immobilized glutathione, a Green fluorescent protein (GFP) tag, a protein that is spontaneously fluorescent and can be bound by nanobodies, a Maltose binding protein (MBP) tag, a protein that binds to amylose agarose, a Nus-tag, a Thioredoxin-tag, an Fc-tag, derived from immunoglobulin Fc domain, allow dimerization and solubilization. A tag that can be used for purification on Protein-A Sepharose; as well as other types of tags such as the Ty tag. Furthermore, the PF4 dominant negative mutants may also include one or more D-amino acids or include chemical modifications such as glycosylation, PEGylation, crosslinking, and the like.

In addition, the polypeptide of this invention comprising at least one of the P1-P12 peptides may be conjugated with a moiety, which may be a protein or non-protein in nature. Such moiety may include a detection moiety capable of emitting a detectable signal (e.g., a optical, radioactive, fluorescent, chemiluminescent signal, and the like) or a solid substrate so as to facilitate detection and/or isolation of the polypeptide.

### B. Chemical Synthesis of Peptides

The amino acid sequences of 12 newly identified cancer-associated peptides termed P1-P12 are provided herein. A polypeptide comprising any one of these peptides thus can be chemically synthesized using conventional peptide synthesis or other protocols well known in the art.

Polypeptides may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an a-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Materials suitable for use as the solid support are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl) phenoxy resin; tert-alkyloxycarbonyl-hydrazidated resins, and the like. Such resins are commercially available and their methods of preparation are known by those of ordinary skill in the art. Briefly, the C-terminal N-α-protected amino acid is first attached to the solid support. The N-α-protecting group is then removed. The deprotected a-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton et al., Solid Phase Peptide Synthesis: A Practical Approach, IRL Press (1989), and Bodanszky, Peptide Chemistry. A Practical Textbook, 2nd Ed., Springer-Verlag (1993)).

### C. Recombinant Expression of Polypeptides

A cancer-associated peptide newly identified in this disclosure having the amino acid sequence set forth in any one of SEQ ID NOs: 1-12 or a polypeptide comprising any one of such peptides can be produced using routine techniques in the field of recombinant genetics, relying on the polynucleotide sequences encoding the peptides disclosed herein.

To obtain high level expression of a nucleic acid encoding a desired polypeptide. one typically subclones a polynucleotide encoding the polypeptide into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. One exemplary eukaryotic expression vector is an adenoviral vector, an adeno-associated vector, or a retroviral vector.

Standard transfection methods can be used to produce bacterial, mammalian, yeast, insect, or plant cell lines that express large quantities of a recombinant polypeptide (e.g., a cancer-associated peptide disclosed herein or a polypeptide comprising an amino acid sequence set forth in any one of SEQ ID NOs: 1-12), which is then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the recombinant polypeptide.

When a recombinant polypeptide is expressed in host cells in satisfying quantity, its purification can follow the standard protein purification procedure including Solubility fractionation, size differential filtration, and column chromatography. These standard purification procedures are also suitable for purifying peptides obtained from chemical synthesis. The identity of the purified peptide may be further verified by methods such as immunoassay's (e.g., Western blot or ELISA) and mass spectrometry.

### III. Diagnosis and Treatment of Cancer

Diagnosis of cancer such as HCC as well as methods of treating or suppressing the progression of cancer and related conditions, are included in the present invention. As illustrated by the present inventors for the first time, the presence of any one or more of the newly identified peptides having the amino acid sequences set forth in SEQ ID NOs: 1-12 is associated with cancer, especially HCC. Thus, a diagnostic method is developed based on the detection of any one or more, e.g., any two or three or more, of such peptides in biological samples taken from a patient being tested for a possible cancerous condition such as a sample comprising circulating blood cells or the pertinent tissues or cells, e.g., a blood sample (such as a whole blood sample or a cellular fraction thereof, for example, all blood cells, lymphocytes and monocytes, or peripheral mononuclear cells or PBMCs) or a liver biopsy. Generally, the detection of the immunogenic peptides of this invention in the MHC complex can be achieved by first isolating the complex from a biological sample, e.g., a blood sample such as a PBMC sample, from a patient being tested due to having suspected clinical symptoms of cancer, e.g., HCC. An affinity-based isolation method is useful for isolation of the complex, for example, an agent with the ability to specifically bind the MHC complex may be used as a “bait” to remove the MHC-immunogenic peptide complex from a suitable sample (e.g., a blood sample) taken from a patient being tested, an environment where many other biomolecules are present. Subsequently, an analytical assay such as mass spectrometry may be employed to detect the presence of any one, two, three or more of the immunogenic peptides of this invention (i.e., P1-P12 having the amnio acid sequences set forth in SEQ ID NOs: 1-12) in the complex.

A variety of methods have been developed based on the mass spectrometry technology to rapidly and accurately quantify target polypeptides even in a large number of samples. These methods involve highly sophisticated equipment such as the triple quadrupole (triple Q) instrument using the multiple reaction monitoring (MRM) technique, matrix assisted laser desorption/ionization time-of-flight tandem mass spectrometer (MALDI TOF/TOF), an ion trap instrument using selective ion monitoring SIM) mode, and the electrospray ionization (ESI) based QTOP mass spectrometer. See, e.g., Pan et al., J Proteome Res. 2009 February; 8(2):787-797.

Detecting the presence of one or more of the cancer-associated peptides of this invention (e.g., at least three of the peptides such as P2, P3, and P10) in a patient sample serves as a preliminary diagnostic indication of cancer, e.g., HCC. At least one subsequent diagnostic method may be used to confirm the diagnosis of the condition, for example, conventional diagnostic methods of blood tests (e.g., to measure liver function), physical examination and imaging tests (e.g., CT scan or MRI to observe any tissue/organ structure anomalies), as well as biopsies of tissue from suspected tumor site may be employed to not only confirm the condition but also to aid devise an appropriate treatment plan.

Upon diagnosis of a suspected condition, e.g., cancer such as HCC, a patient may receive treatment in accordance with the attending physician's determination of treatment plan, depending on the specific factors in the patient's medical and physical condition as well as the etiology, pathology, and severity of the cancer-related condition. For instance, surgical intervention may be used to remove significant tumor burden, possibly in combination with other means of cancer treatment such as radiation therapy and/or immunotherapy.

### IV. Pharmaceutical Compositions and Administration

The present invention also provides pharmaceutical compositions comprising an effective amount of at least one, potentially two or three or more, of the immunogenic peptides P1-P12 of this invention for eliciting a desirable immune response, therefore useful in both prophylactic and therapeutic applications designed for various diseases and conditions involving undesired cellular proliferation, especially malignancies including liver cancer such as HCC. Pharmaceutical compositions of the invention are suitable for use in a variety of drug delivery systems. Suitable formulations for use in the present invention are found in Remington's Pharmaceutical Sciences, Mack Publishing Company, Philadelphia, PA, 17th ed. (1985). For a brief review of methods for drug delivery, see, Langer, Science 249:1527-1533 (1990).

The pharmaceutical compositions of the present invention can be administered by various routes, e.g., oral, nasal, subcutaneous, transdermal, intramuscular, intravenous, or intraperitoneal. The routes of administering the pharmaceutical compositions include systemic or local delivery to a subject suffering from a condition exacerbated by inflammation at daily doses of about 0.01-5000 mg, preferably 5-500 mg, of any one of the immunogenic peptides of this invention as described herein for a 70 kg adult human per day. The appropriate dose may be administered in a single daily dose or as divided doses presented at appropriate intervals, for example as two, three, four, or more subdoses per day.

For preparing pharmaceutical compositions containing an immunogenic peptide, inert and pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., an immunogenic peptide of this invention. In tablets, the active ingredient (the immunogenic peptide) is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound of an immunogenic peptide of this invention with encapsulating material as a carrier providing a capsule in which the mutant (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., an immunogenic peptide of the present invention) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., an immunogenic peptide of this invention) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions containing the active ingredient can be administered for prophylactic and/or therapeutic treatments. In therapeutic applications, compositions are administered to a patient already suffering from a condition that may be exacerbated by inappropriate or undesirable cellular proliferation in an amount sufficient to prevent, cure, reverse, or at least partially slow or arrest the symptoms of the condition and its complications. An amount adequate to accomplish this is defined as a “therapeutically effective dose.” Amounts effective for this use will depend on the severity of the disease or condition and the weight and general state of the patient, but generally range from about 0.1 mg to about 2,000 mg of the immunogenic peptide per day for a 70 kg patient, with dosages of from about 5 mg to about 500 mg of the mutant polypeptide per day for a 70 kg patient being more commonly used.

In prophylactic applications, pharmaceutical compositions containing the active ingredient are administered to a patient susceptible to or otherwise at risk of developing a disease or condition involving inappropriate or undesirable cellular proliferation in an amount sufficient to delay or prevent the onset of the symptoms. Such an amount is defined to be a “prophylactically effective dose.” In this use, the precise amounts of the immunogenic peptide again depend on the patient's state of health and weight, but generally range from about 0.1 mg to about 2,000 mg of the peptide for a 70 kg patient per day, more commonly from about 5 mg to about 500 mg for a 70 kg patient per day.

Single or multiple administrations of the compositions can be carried out with dose levels and pattern being selected by the treating physician. In any event, the pharmaceutical formulations should provide a quantity of a compound sufficient to effectively alleviate at least one pertinent symptoms associated with the target condition/disease in the patient, either therapeutically or prophylactically.

For a formulation used in situations where the intended therapeutic effects are achieved or enhanced via a desired immune response elicited by the administration of such formulation, at least one excipient capable of promoting or stimulating the immune response, such as an adjuvant, may be included in the formulation.

### V. Therapeutic Applications Using Nucleic Acids

A variety of diseases and conditions involving undesirable or inappropriate cellular proliferation can be treated by therapeutic approaches that involve introducing into a patient's body, including to a targeted tissue or cells, a nucleic acid encoding an immunogenic peptide (e.g., any one of P1-P12 or a larger polypeptide comprising the amino acid sequence set forth in any one of SEQ ID NOs: 1-12) such that the expression of the peptide leads to reduced or abolished symptoms of a relevant condition. Those amenable to treatment by this approach include a broad spectrum of conditions involving inappropriate and/or undesirable cellular proliferation. For discussions on the application of gene therapy towards the treatment of genetic as well as acquired diseases, see, Miller Nature 357:455-460 (1992); and Mulligan Science 260:926-932 (1993).

### A. Vectors for Nucleic Acid Delivery

For delivery to a cell, a tissue, or an organism such as a patient, a polynucleotide sequence encoding a peptide comprising or consisting of any one of the immunogenic peptides P1-P12 of the invention can be first incorporated into a vector. Examples of vectors used for such purposes include expression plasmids capable of directing the expression of the such a peptide in the target cell. In other instances, the vector is a viral vector system wherein the polynucleotide is incorporated into a viral genome that is capable of transfecting the target cell. In a preferred embodiment, the polynucleotide sequence encoding the peptide can be operably linked to expression and control sequences that can direct transcription of sequence in the desired target host cells. Typically, the polynucleotide coding sequence is operably linked to a heterologous promoter sequence, which drives transcription of the coding sequence in the target cells/tissue/organism. Thus, one can achieve intended therapeutic effects under appropriate conditions in the target cells/tissue/organism.

### B. Gene Delivery Systems

As used herein, “gene delivery system” refers to any means for the delivery of a nucleic acid of the present invention to a target cell. The nucleic acid may be in the form of a DNA or RNA molecule, either single-stranded or double-stranded. Optionally, the DNA or RNA may contain artificial or non-natural nucleotide residue(s) in addition to naturally-occurring nucleotides. For delivering a DNA molecule, a variety of vectors may be used, including plasmids and viral vectors. Viral vector systems useful in the introduction and expression of a nucleic acid include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the polynucleotide sequence encoding a peptide of this invention is inserted into such vectors to allow packaging of the gene construct, typically with accompanying viral DNA, followed by infection of a sensitive host cell and expression of the peptide of interest.

Similarly, viral envelopes used for packaging gene constructs that include the polynucleotide sequence can be modified by the addition of receptor ligands or antibodies specific for a receptor to permit receptor-mediated endocytosis into specific cells (see, e.g., WO 93/20221, WO 93/14188, and WO 94/06923).

Retroviral vectors may also be useful for introducing the nucleic acid of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712, Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired polynucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired polynucleotide sequence. As a result, the patient is capable of expressing, for example, the desired peptide encoded by the polynucleotide sequence, thus eliminating or reducing one or more symptoms associated with the target disease or condition, including cancer such as HCC.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

### C. Pharmaceutical Formulations

When used for pharmaceutical purposes, the nucleic acid to be delivered is generally formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can further include a stabilizer, an enhancer, and/or other pharmaceutically acceptable carriers or vehicles. A pharmaceutically acceptable carrier can contain a physiologically acceptable compound that acts, for example, to stabilize the inhibitory nucleic acids of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, PA, 17th ed. (1985).

Similar to a polypeptide-based formulation intended for use to elicit a desired immune response, at least one excipient capable of promoting or stimulating the immune response, such as an adjuvant, may be included in the formulation for nucleic acid delivery. In addition, other ingredients such as lipid nanoparticles (LNPs) may also be included to further improve delivery and therefore therapeutic efficacy.

### D. Administration of Formulations

The formulations containing a nucleic acid of this invention (e.g., encoding any one of P1-P12 peptides) can be delivered to any tissue or organ using any delivery method known to the ordinarily skilled artisan. In some embodiments of the invention, the nucleic acid is formulated in mucosal, topical, and/or buccal formulations, particularly mucoadhesive gel and topical gel formulations. Exemplary permeation enhancing compositions, polymer matrices, and mucoadhesive gel preparations for transdermal delivery are disclosed in U.S. Pat. No. 5,346,701.

The formulations containing the nucleic acid are typically administered to a cell. The cell can be provided as part of a tissue or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acid is introduced into cells by such methods as microinjection, calcium phosphate precipitation, liposome fusion, ultrasound, electroporation, or biolistics. In further embodiments, the nucleic acid is taken up directly by the tissue of interest. In some cases, the use of lipid nanoparticles may enhance the rate of delivery and cellular uptake.

In some embodiments of the invention, the nucleic acid is administered ex vivo to cells or tissues explanted from a patient, then returned to the patient. Examples of ex vivo administration of therapeutic gene constructs include Nolta et al., Proc Natl. Acad. Sci. USA 93(6); 2414-9 (1996); Koc et al., Seminars in Oncology 23 (1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93 (1):402-6 (1996).

Effective dosage of the formulations will vary depending on many different factors, including means of administration, target site, physiological state of the patient, and other medicines administered. Thus, treatment dosages will need to be titrated to optimize safety and efficacy. In determining the effective amount of the vector to be administered, the physician should evaluate the particular nucleic acid used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector. To practice the present invention, doses ranging from about 10 ng-1 g, 100 ng-100 mg, 1 μg-10 mg, or 30-300 μg of the nucleic acid per patient are typical. Doses generally range between about 0.01 and about 50 mg per kilogram of body weight, preferably between about 0.1 and about 5 mg/kg of body weight or about 10⁸-10¹⁰ or 10¹² viral particles per injection. In general, the dose equivalent of a naked nucleic acid from a vector is from about 1 μg-100 μg for a typical 70 kg patient, and doses of vectors which include a retroviral particle are calculated to yield an equivalent amount of the nucleic acid.

### VI. Anti-Cancer Therapeutic Agents

Previously known therapeutic agent or agents with anti-cancer efficacy may be used in combination with an immunogenic peptide or a nucleic acid encoding the peptide as described herein during the practice of the present invention for the purpose of effectively treating cancer. In such applications, one or more of these previously known effective anti-cancer therapeutic agents can be administered to patients concurrently with an effective amount of the formulation containing at least one peptide or encoding nucleic acid either together in a single composition or separately in two or more different compositions. They may be used in combination with the active agent of the present invention (e.g., an immunogenic peptide or an encoding nucleic acid) to suppress cancer growth, inhibit cancer metastasis, and facilitate remission from the disease.

For example, various chemotherapeutic agents are known to be effective for use to treat various cancers. As used herein, a “chemotherapeutic agent” encompasses any chemical compound exhibiting suppressive effect against cancer cells, thus useful in the treatment of cancer. Classes of chemotherapeutic agents include, but are not limited to: alkylating agents, antimetabolites, kinase inhibitors, spindle poison plant alkaloids, cytoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods disclosed herein also include cytostatic and/or cytotoxic agents.

Exemplary anti-cancer therapeutic agents include alkylating agents such as altretamine, bendamustine, busulfan, carboquone, carmustine, chlorambucil, chlormethine, chlorozotocin, cyclophosphamide, dacarbazine, fotemustine, ifosfamide, lomustine, melphalan, melphalan flufenamide, mitobronitol, nimustine, nitrosoureas, pipobroman, ranimustine, semustine, streptozotocin, temozolomide, thiotepa, treosulfan, triaziquone, triethylenemelamine, trofosfamide, and uramustine; anthracyclines such as aclarubicin, daunorubicin, doxorubicin, epirubicin, idarubicin, mitoxantrone, pirarubicin, valrubicin, and zorubicin; cytoskeletal disruptors (taxanes) such as abraxane, cabazitaxel, docetaxel, larotaxel, paclitaxel, taxotere, and tesetaxel; epothilones such as ixabepilone; histone deacetylase inhibitors such as vorinostat, romidepsin, and inhibitors of topoisomerase I such as belotecan, camptothecin, exatecan, gimatecan, irinotecan, and topotecan; inhibitors of topoisomerase II such as etoposide, teniposide, and tafluposide; kinase inhibitors such as bortezomib, erlotinib, gefitinib, imatinib, vemurafenib, and vismodegib; nucleotide analogs and precursor analogs such as azacitidine, azathioprine, capecitabine, cytarabine, doxifluridine, fluorouracil, gemcitabine, hydroxyurea, mercaptopurine, methotrexate, and tioguanine (formerly thioguanine); peptide antibiotics such as actinomycin and bleomycin; platinum-based agents such as carboplatin, cisplatin, dicycloplatin, oxaliplatin, nedaplatin, and satraplatin; retinoids such as alitretinoin, bexarotene, and tretinoin; and vinca alkaloids and derivatives such as vinblastine, vincristine, vindesine, and vinorelbine.

Immunotherapeutic approaches useful for cancer treatment include (1) active immunotherapy, which directs the immune system to specifically target the cancer cells, e.g., targeted antibody therapy and cell-based immunotherapy such as CAR T cell therapy; and (2) passive immunotherapy, e.g., using checkpoint inhibitors and cytokines to stimulate the immune system without specifically targeting cancer cells. Various monoclonal antibodies are used in targeted antibody therapy. Examples of such antibodies and their conjugates include adotrastuzumab (HER2), alemtuzumab (CD52), bevacizumab (VEGF), brentuximab (CD30), capromab (PSMA), cetuximab (EGFR), elotuzumab (SLAMF7), ibritumomab; (CD20), necitumumab (EGFR), obinutumab (CD20), ofatumumab (CD20), olaratumab (PDGFRA), panitumumab (EGFR), pertuzumab (HER2), ramucirumab (VEGFR2), rituximab (CD-20), trastuzumab (HER-2), inotuzumab-ozogamicin (CD22), gemtuzumab-ozogamicin (CD33), and bevacizumab-awwb (VEGF). Currently approved checkpoint inhibitors target molecules CTLA4, PD-1, and PD-L1, including ipilimumab (CTLA4), nivolumab, pembrolizumab, cemiplimab, spartalizumab (PD-1), atezolizumab, avelumab, and durvalumab (PD-L1). Cytokines for use in the treatment of cancer and associated conditions include granulocyte colony-stimulating factor (G-CSF), granulocyte macrophage colony-stimulating factor (GM-CSF), interleukin-2 (IL-2), and interleukin-11 (IL-11).

### VII. Kits

The invention also provides kits for preventing or treating a condition involving undesirable/excessive cellular proliferation including cancers of different types through the administration to a patient in need thereof a composition of the present invention. The kits typically include a first container that contains a pharmaceutical composition comprising an effective amount of at least one of the peptides of this invention, optionally with a second or third, or additional container, each containing a second, third, or additional composition that comprises a second, third, or additional peptide. Furthermore, the kits may include another container containing one or more anti-cancer therapeutic agents, such as those named in the last section.

In some cases, the kits will also include informational material containing instructions on how to dispense the pharmaceutical composition, including description of the type of patients who may be treated (e.g., a person suffering from or at risk of a disease or condition involving undesirable or excessive cellular proliferation, including various types of cancer such as HCC), the schedule (e.g., dose and frequency of administration) and route of administration, and the like.

### EXAMPLES

The following examples are provided by way of illustration only and not by way of limitation. Those of skill in the art will readily recognize a variety of non-critical parameters that could be changed or modified to yield essentially the same or similar results.

### Introduction

Hepatocellular carcinoma (HCC) is a highly prevalent form of primary liver cancer, considered a leading cause of cancer-related mortality, and associated with one of the lowest survival rates (1). In the past decades, immune checkpoint blockades (ICB) of immunosuppressive surface proteins have shown promising clinical responses in the treatment of HCC (2-4). A prerequisite of ICB anti-tumor immune responses is the tumor-specific effector T-cell recognition of tumor-associated antigen (TAA) and tumor-specific antigen (TSA) displayed by human major histocompatibility complex (MHC) molecules on the tumor surface. Recent studies have suggested that transcripts not annotated as protein-coding, such as frame-shift products, lncRNAs, and untranslated regions (UTRs), represent reservoirs of MHC-I-associated antigens (5-10). To discover novel transcript variants associated with HCC carcinogenesis, we employed a hybrid transcriptomic profiling approach that combined SMRT-seq long-read and Illumina short-read RNA-seq to profile the transcript variants landscape in in-house patient-derived HCC cell lines (11). By searching this in-house database against the immunopeptidome generated in our study, we successfully identified 12 novel peptide sequences derived from previously unannotated open reading frames (ORFs). Through In-silico prediction and subsequent experimental validation, we confirmed the immunogenicity of these 12 novel peptides, thereby highlighting their potential therapeutic values. These findings provide evidence for immunogenic peptides translated from supposedly non-coding RNA sequences, thus opening up novel options for HCC therapy development.

### Material and Methods

### Cell Culture

Patient-derived HCC cell cultures (HKCI-2, HKCI-4, HKCI-9, HKCI-10, HKCI-11, HKCI-C1, and HKCI-C2) were maintained in AIM-V medium (Gibco, Life Technologies). Immortalized human hepatocyte cell line MIHA was cultured in DMEM medium. All medium was supplemented with 10% fetal bovine serum (FBS), 2 mM L-glutamine (Life Technologies), and 100 U/mL of Penicillin-Streptomycin (Life Technologies). The cells were cultured in a 37° C., 5% CO₂ incubator for three days until 90% confluence for subsequent MHC immunoaffinity purification.

### Human Blood Specimens

We obtained peripheral mononuclear cells (PBMCs) from healthy donor buffy coat samples via density gradient centrifugation. We utilized QIAamp DNA Blood Kits (Qiagen) to isolate DNA for HLA typing with LinkSeq™ HLA-ABCDRDQDP+ 384 Kit (One Lambda) on a QuantStudio™ 7 Flex Real-Time PCR System. Human T and dendritic cells (DCs) were enriched from PBMCs using a CD8 cell isolation kit and Pan-DC enrichment kit (Miltenyi Biotec), respectively, and subsequently cultured in RPMI-1640 medium (Life Technologies) supplemented with 10% FBS and 100 U/mL of Penicillin-Streptomycin.

### Affinity Purification of HLA Class I Molecules and Enrichment of HLA-Bound Peptides

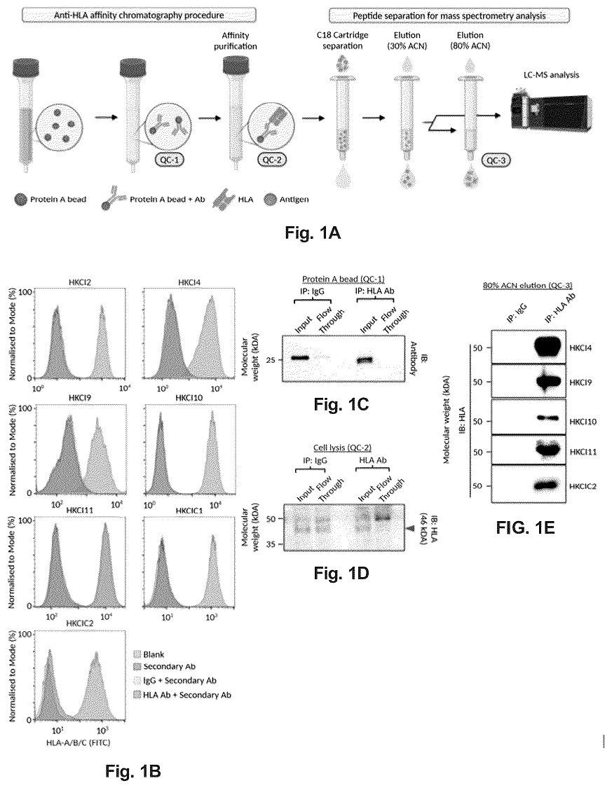

To generate the HLA class I immunoaffinity column. Ultra-LEAFTM Purified anti-human HLA-A/B/C antibody (BioLegend, clone W6/32) and Ultra-LEAFTM Purified Mouse IgG2a K isotype antibody (BioLegend, clone MOPC-173) were incubated and crosslinked with Protein A Sepharose™ 4B beads (Invitrogen), respectively. Cell lysate supernatant was generated from freshly prepared cells by homogenizing in lysis buffer (1% NP-40, 150 mM NaCl, proteinase inhibitor. 50 mM Tris, pH 8.0) followed by ultracentrifuge. After pre-clearing, the lysate was incubated with an immunoaffinity column for 16 hours at 4° C. Immunoaffinity column was washed following the protocol described in (12). HLA-antigen complex was eluted with 0.2% trifluoracetic acid. Antigen peptides were further separated from HLA molecules on a Sep-Pak tC18 Plus Light Cartridge (Waters) with 30% acetonitrile in 0.1% trifluoroacetic acid and subsequently lyophilized with CentriVap vacuum concentrator (Lanconco) for mass spectrometry analysis (FIG. 1a). Chemicals and solvents used in the wash and elution steps were in LC-MS grade.

### Liquid Chromatography-Mass Spectrometry (LC-MS/MS) Analysis of MHC Class I Antigen Peptides

The Dionex Ultimate3000 nanoRSLC system, coupled with a Thermo Fisher Orbitrap Fusion Tribid Lumos mass spectrometer, was used to analyze eluted antigen peptides. Peptides were separated on a commercial C18 column coupled to a NanoTrap column (Thermo Fisher). Separation was attained using a linear gradient of increasing buffer B (80% acetonitrile and 0.1% formic acid) and declining buffer (0.1% formic acid) at 300 nL/minute. The mass spectrometer was operated in positive polarity mode with a capillary temperature of 300° C. Full MS survey scan resolution was set to 120,000 with an automatic gain control (AGC) target value of 5×10⁴, maximum ion injection time (IT) of 50 ms, and a scan range of 350-1500 m/z. Charge states from 1+ to 4+ were acquired. The dependent scan on single charge state per precursor only setting was also enabled. A data-dependent top speed method with a time interval of 2 seconds between every survey scan was operated, during which higher-energy collisional dissociation (HCD) was used. MS/MS spectra were obtained at 60,000 MS² resolution with a normalized AGC target of 200% and maximum ion injection time (IT) of 100 ms, 1.6 m/z isolation width, and normalized collisional energy of 30. Preceding precursor ions targeted for HCD were dynamically excluded for 10 seconds. All solvents were equivalent to or higher than LC-MS grade.

### Proteomics Database Construction From Transcriptomic Data

We utilized the SUPPA2 analysis with proportion spliced-in (PSI) method to identify alternative transcripts from the short-read RNA-Seq data. Tumor-specific transcripts were extracted by removing transcripts with zero PSI in MIHA and positive PSI in at least one patient-derived cell line (13). To detect fusion transcripts between transposable elements (TE) and exons, we aligned the short reads using STAR chimeric alignment and assembled the transcripts using Stringtie (14,15). Afterward, we compared the resulting transcript assembly with TE and exon coordinates using the custom scripts provided in reference (16). The Iso-Seq clustering workflow and IsoSeq3 refine step were utilized to analyze the long-read RNA-Seq data. Consensus sequence clustering was performed on the resulting full-length reads to ease isoform identification (17). The Iso-Seq clusters were subjected to splice-aware alignment using Minimap2, followed by Stringtie transcriptome assembly. TE-derived alternative transcripts were identified using TEprof2 (18). From the alternative transcripts identified from long-read and short-read data, the ORFik R package detected open reading frames (ORFs) in alternative transcripts that commence with ATG and are at least 25 amino acids long (19). These ORFs were subsequently translated into a non-reference proteomics database using the Biostrings Bioconductor package (20).

### Mass Spectrometry Data Analysis

The mass spectrometry (MS) data was analyzed using version 2.1.4.0 of the MaxQuant computational proteomics platform, employing the Andromeda search engine (21). The search was performed against the human SwissProt database, and the customized non-reference proteomics database mentioned earlier. The MaxQuant search parameters included unspecific digestion, no fixed modifications, variable modifications of methionine oxidation and N-terminal acetylation, and a peptide length range of 8-15. The peptide-spectrum matches (PSMs) were subjected to statistical filtering using a peptide false discovery rate (FDR) of 0.01 and a protein FDR of 1. Subsequent quality control was carried out by the computational pipeline Proteomics Quality Control (PXTQC) (22). Several filtering criteria were applied, including removing contaminants and reversed decoy sequences, a minimum MS/MS count of 1, and a maximum posterior error probability (PEP) threshold of 0.01. The hydrophobicity of the filtered peptides was estimated using the R package alakazam (23, 24). Non-canonical peptide candidates were selected from amino acid sequences exclusively detected in the customized database rather than the UniProt database. The quality of the PSMs of non-canonical peptides was confirmed by spectrum visualization using the proteomics data viewer PDV (25).

### HLA and T-Cell Receptor Affinity Prediction

We filtered the peptides identified from MS using negative controls. These filtered peptides were then compared against patient-specific HLA alleles (4-digit resolution) determined through HLA typing, enabling peptide-HLA affinity prediction by the artificial neural network NetMHCPan (version 4.1). The HLA binding potential of the query peptide was ranked against the dataset trained with MS Eluted Ligands (EL) peptides or quantitative Binding Affinity (BA). If the best ranking percentile fell between 0.5 and 2, it would be considered a weak HLA-binder. To qualify as a strong binder for the corresponding patient, the best-ranking percentile of a peptide must be below 0.5 (26). To understand the underlying HLA binding patterns of cell-line specific peptides, we employed GibbsCluster 2.0 to cluster peptides with consensus binding motifs (27). The resulting clusters were validated against known binding motifs recorded in the MHC motif viewer, with consensus motifs visualized using Seq2Logo (28, 29). We queried all strong and weak binding interactions predicted from EL or BA against DeepImmuno to evaluate T-cell activities (30). The resulting DeepImmuno score indicated the potential for interactions between T-cell receptors (TCRs) and the input peptide-HLA complex.

### Characterization of Non-Canonical Peptides

We extracted the sequences of non-canonical peptides and their respective ORFs from the MaxQuant peptide output file. We used the Salmon software to quantify the ORF expression levels both among tested cell lines and 10 randomly selected normal liver samples from the Genotype-Tissue Expression (GTEx) cohort (31, 32). We also searched for exact peptide sequence matches in the corrected long-read bam files. The candidates' expression or absence/presence was visualized using the Complex Heatmap R package (33). We also employed the Integrated Genome Viewer to visualize the genomic coordinates of the non-canonical peptides and ORFs (34).

### Enzyme-Linked Immunospot (ELISpot) Assay to Determine Peptide Immunogenicity

Dendritic cells (DCs) were pulsed with a 10 μg mL⁻¹ peptide concentration at a cell density of 10⁵ cells/mL for 4 hours at 37° C. Following this, the DCs were mixed with autologous CD8+ T cells at a DC:T cell ratio of 1:10. The cell mixture was then aliquoted into an ELISpot plate at 100 μL per well and incubated for 24 hours. The secreted IFN-γ signal was detected using the ELISpot Plus Human IFN-γ (ALP) kit (Mabtech). All peptides used in this study were synthesized by GenScript Biotech and had a purity greater than 98%. The trifluoroacetic acid residue was less than 1%, and the endotoxin level was at most 0.01 EU/μg. In addition to the anti-CD3 antibody, we utilized NY-ESO-1 as one of the positive controls. To eliminate false-positive immune signals from potential contaminants during peptide synthesis, we synthesized a peptide derived from human Actin within the amino acid region 1-9 (Actin1-9) as a negative control. The amino acid sequences of NY-ESO-1 and Actin1-9 are SLLMWITQV and MCDEDETTA, respectively. The ELISpot analysis presented the IFN-γ count as the mean±standard deviation (SD). Statistical analysis was performed using an unpaired Student's t-test, which defined the statistical significance as p-values<0.05. We conducted all statistical analyses using Prism software version 10 (GraphPad).

### Comparisons Against Public Datasets

To investigate the expression levels of translational regulators, we utilized the GEPIA2 web server to compare RNA expression data of tumor and non-tumor liver samples from The Cancer Genome Atlas (TCGA) and GTEx GEPIA2 (35). To validate and understand the underlying mechanisms responsible for p1-p12 translation, the ORF and peptide sequences of p1-p12 were queried against m6A-seq and ribo-seq data using the following databases: m6A-Atlas v2.0, REPIC, RMBase v2.0, Ribo-uORF, and SMProt (35-39).

### Results

### Purification of HCC Neoantigen by MHC-Based Affinity Chromatography

Before conducting HLA-based affinity purification, we evaluated the surface HLA-A/B/C expression in patient-derived HCC cell lines using flow cytometry. Analysis of the mean fluorescence intensity showed a significant signal in all HCC cell lines stained with HLA-A/B/C antibody (clone W6/32) compared with isotype control, indicating that all seven cell lines were MHC class I-positive suitable for systematic profiling of tumor-associated antigens (FIG. 1b). Next, we performed PCR-based HLA typing to define the HLA alleles in each cell line to enable downstream immunogenic profiling. To improve the detection rate of presented epitopes by MS, 1.9×10⁹ cells were used in each affinity purification. In addition, three quality control steps ensured equal loading of IgG/antibody, maximum binding of antigen-HLA complexes, and sufficient antigen elution from the column (FIG. 1c-e).

### Overview of MHC-affinity Mass Spectrometry Data

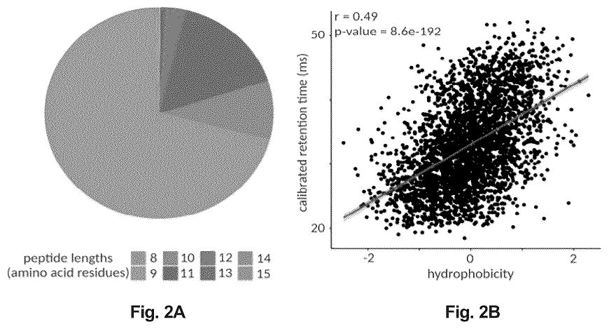

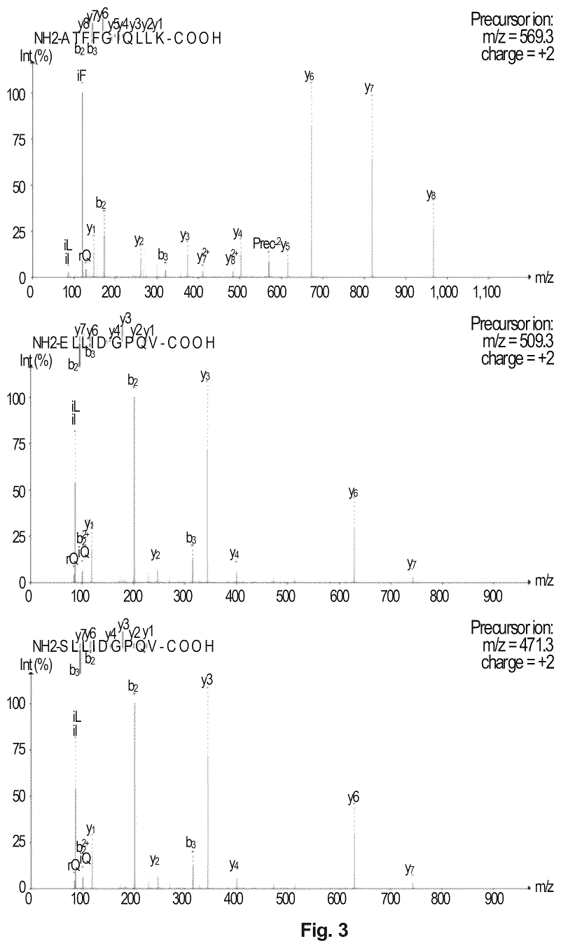

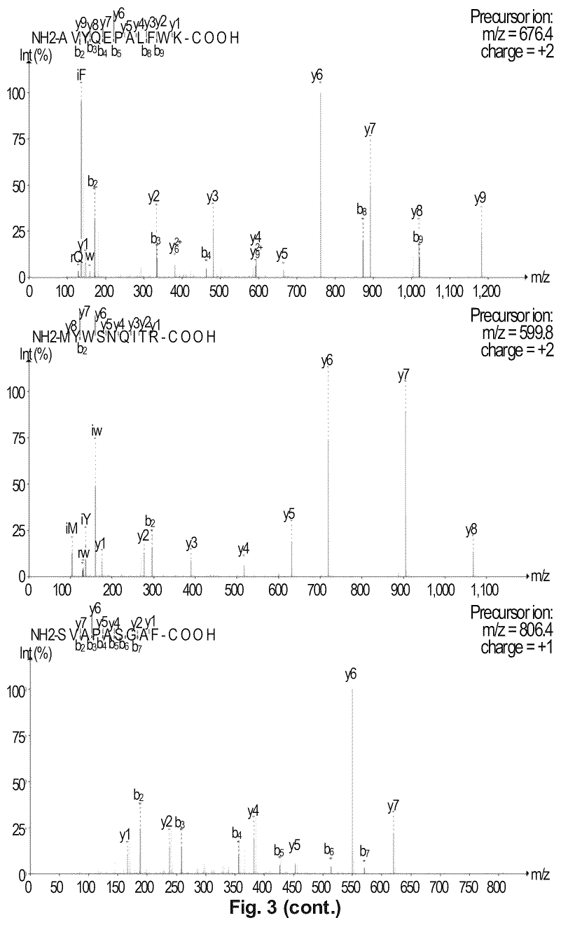

Canonical and non-canonical peptides were identified from seven patient-derived cell lines using raw data from MHC-pulldown mass spectrometry. Nearly 75% of peptides were nine amino acids long, as expected from HLA-I enriched peptides (FIG. 2a). The predicted hydrophobicity showed a generally positive correlation with retention time in the liquid chromatography column (FIG. 2b), further supporting the reliability of peptide identification by MaxQuant. The filtering step resulted in the retention of 3141 peptides, encompassing 26 Cancer-Testis Antigen (CTA) peptides and 12 non-canonical peptides (Table 1). The theoretical spectra of the non-canonical peptides match the experimental spectra, further ensuring the reliability of the non-canonical peptide identification (FIG. 3).

TABLE 1
The amino acid sequences, naming and
characteristics of all identified non-
canonical peptides

| | Short | | Associated |
|---|---|---|---|
| Sequence | form | Classification | gene |
| ATFFGIQLLK (SEQ ID NO: 1) | p1 | 5'-UTR | RSPRY1 |
| AVYQEPALFWK (SEQ ID NO: 2) | p2 | 5'-UTR | SERPINB6 |
| EVVTEVVSR (SEQ ID NO: 3) | p3 | 5'-UTR | ZNF32 |
| MAEDYWDGRLR (SEQ ID NO: 4) | p4 | 5'-UTR | CDK16 |
| MYWSNQITR (SEQ ID NO: 5) | p5 | 5'-UTR | NUDT1 |
| NTFNTSLLY (SEQ ID NO: 6) | p6 | CDS | TMEM68 |
| | | (frame-shift) | |
| QSPVALRPL (SEQ ID NO: 7) | p7 | CDS | CIAO2A |
| | | (frame-shift) | |
| SLLIDGPQV (SEQ ID NO: 8) | p8 | IncRNA | LINC02085 |
| STIHLASQFTK (SEQ ID NO: 9) | p9 | CDS | CRYBG1 |
| | | (frame-shift) | |
| SVAPASGAF (SEQ ID NO: 10) | p10 | 5'-UTR | PLOD3 |
| TEDPETSVL (SEQ ID NO: 11) | p11 | 5'-UTR | DNAJC22 |
| VLPDQHLVTTV (SEQ ID NO: 12) | p12 | IncRNA | PPP1R14B-AS1 |

### Overall Patient-Specific MHC Affinity

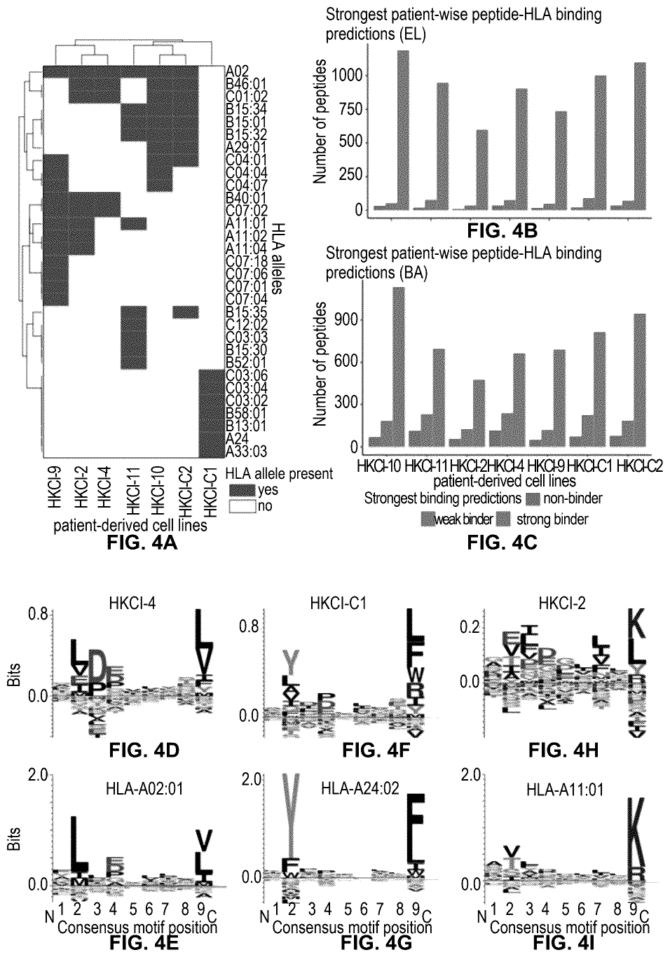

We employed PCR-based LinkSēq™ HLA typing to identify the HLA alleles in each HCC cell line (FIG. 4a). The peptide-HLA affinities were predicted by NetMHCPan, using cell-line specific MHC-bound peptides as queries against all HLA-alleles of the same cell line. The results showed that most peptides exhibited affinity to at least one host HLA allele, with strong binders representing the most significant proportion (FIG. 4b-c). Such patterns confirm the immunogenic potential of the MHC-bound peptides in this study. We further compared the patient-specific immunopeptidome in this study against established HLA allele motifs. Overall, the consensus motifs agree with pre-existing data (FIGS. 4d-4i). For example, the consensus motif from HKCI-4 exhibited a leucine in the second position and leucine/valine in the ninth position (FIG. 4d). These patterns align with the consensus motif of the HLA-A02:01 peptidome (FIG. 4e). The most notable feature of the HKCI-C1 consensus motif is the tyrosine and phenylalanine in the second and ninth positions, respectively (FIG. 4f). This pattern might come from HLA-A24. which exhibits the same tyrosine and phenylalanine enrichment pattern (FIG. 4g).

### Visualization and Prioritization of Non-Canonical Peptides

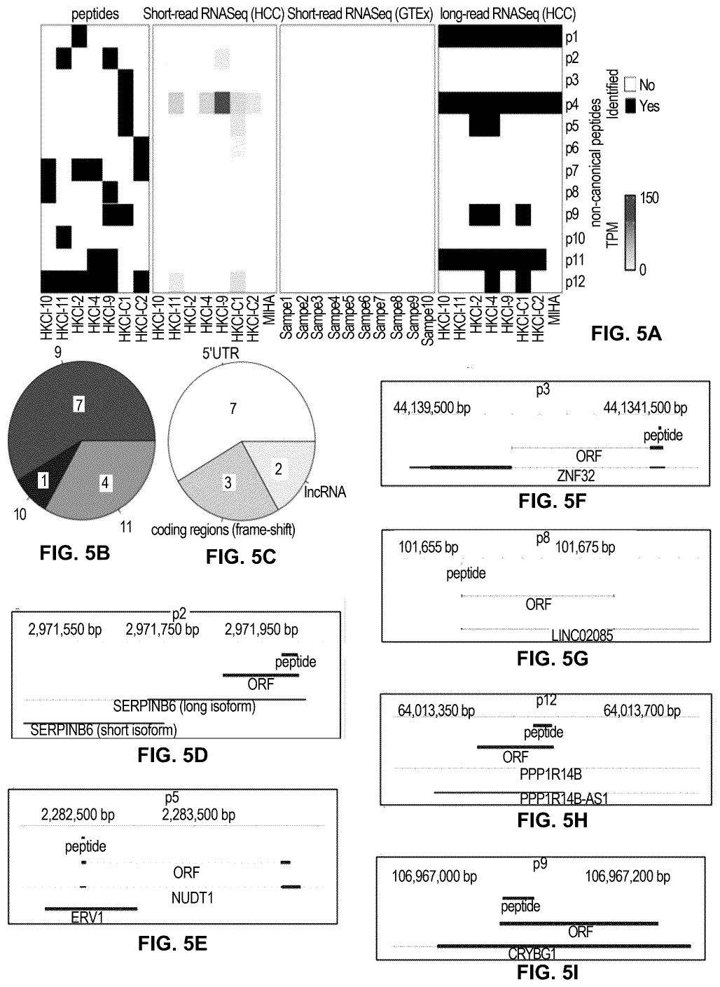

We investigated the expression levels of the non-canonical peptides at the peptide. short-read RNA, and long-read RNA levels. We included 10 GTEx liver samples and an immortalized human hepatocyte line MIHA as negative controls (FIG. 5a). Our results demonstrated that each HCC cell line expressed at least three non-canonical peptides. While some peptides were only present in one sample, others, such as p12, showed recurrent translation across samples. Some ORFs, including those responsible for p2, p4, p5, p6, and p12, demonstrated tumor-specificity at the ORF short-red RNA expression. Furthermore, peptides like p9 and pll were detectable in the long-read data for cancer cell lines but not in MIHA. Conversely, the ten randomly selected liver GTEx samples exhibited almost negligible expression, with TPM values close to zero for most measurements.

The distribution of peptide lengths was similar to the rest of the peptidome, with over half of the peptides being nine amino acids long (FIG. 5b). The 5′-UTRs (untranslated regions) encoded seven of twelve candidate peptides. The other two peptides were products of long non-coding RNAs (lncRNAs) cryptic translation (FIG. 5c and Table 1). One of these peptides, p2, was derived from an extended 5′ end of a long isoform of SERPINB6 that displayed limited non-tumor expression across livers and other tissue types (FIG. 5d). Furthermore, we found that peptide p5. another 5′-UTR-derived candidate, was part of a long-terminal region annotated as ERVI (FIG. 5e). Peptide p3 originated from the 5′-UTR of the ZNF32 gene, with the open reading frame spanning across a splice junction (FIG. 5f). Notably, peptide p8 was derived from an immunoregulatory lncRNA LINC02085. indicating a potential dual immunoregulatory role at both the RNA and peptide levels (FIG. 5g). Additionally, we identified another lncRNA-encoded peptide, p12,which is antisense to PPPIR4B (FIG. 5h). Meanwhile, p9 is a frame-shifted CDS-derived peptide with a modest expression level in GTEx liver tissues compared to other tissue types, such as the esophagus (FIG. 5i). Overall, our findings underscore the diverse origins and characteristics of the peptides in our study.

### Experimental Evaluation of Candidate Peptides Immunogenicity

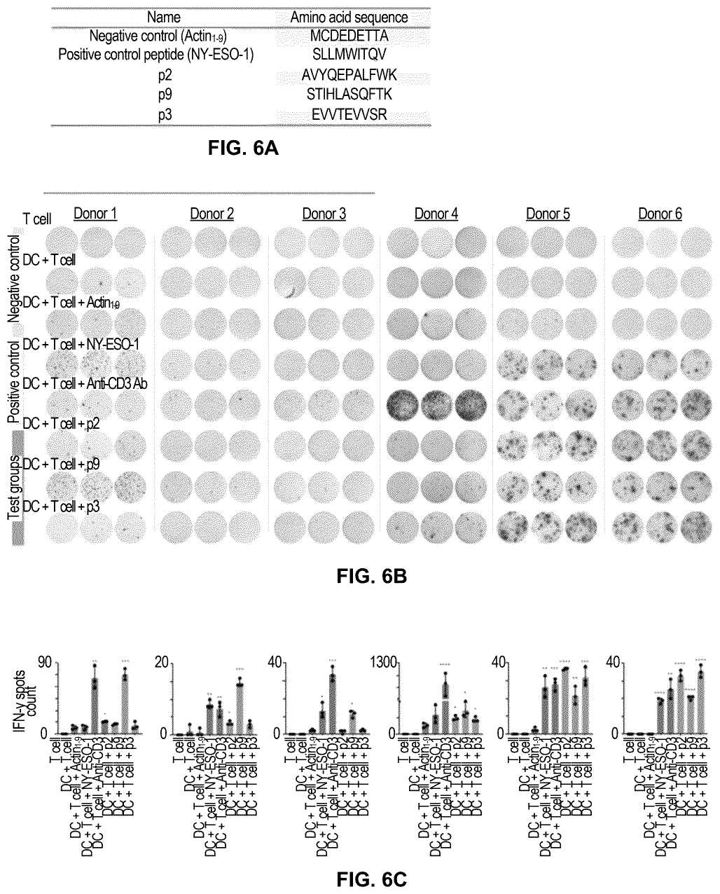

We utilized ELISpot assay, which quantifies cytokine IFN-γ releasing by CD8⁺ T lymphocytes isolated from PBMCs of healthy subjects, to verify the immunogenic potential of candidate peptides. In our selection of peptides for the ELISpot assay, we aimed to cover various genomic characteristics and obtain evidence for tumor-specificity. Specifically, we chose p2 due to its origin from an extended 5′-UTR and its tumor-specificity based on short-read data. Additionally, we included p9 as it represents a tumor-specific frame-shift product with validation from long-read sequencing. Furthermore, we selected p3 to exemplify junction-spanning ORFs (FIG. 6a). We conducted an ELISpot analysis using immune cells isolated from six healthy donors to evaluate the immunogenic potential of the candidate peptides. We co-cultured pan-DCs loaded with the candidate peptides with autologous CD8′ T cells for 24 hours and assessed the secretion of IFN-γ as an indicator of T-cell activation and immunogenicity. Our results demonstrated that NY-ESO-1 triggered a significant T cell immune response in five out of six donor samples compared to the unstimulated control. Actin₁₋₉ peptide generated a negligible response even at a concentration of 1 ug mL⁻¹, indicating the reliability of our assay in quantifying antigen-dependent CD8 T cell responses (FIG. 6b-c). Our findings revealed that three candidate peptides induced significantly stronger T cell activation over Actin₁₋₉ in at least three of six donors (FIGS. 6b-6c).

### Computational Prediction of HLA and TCR Affinities

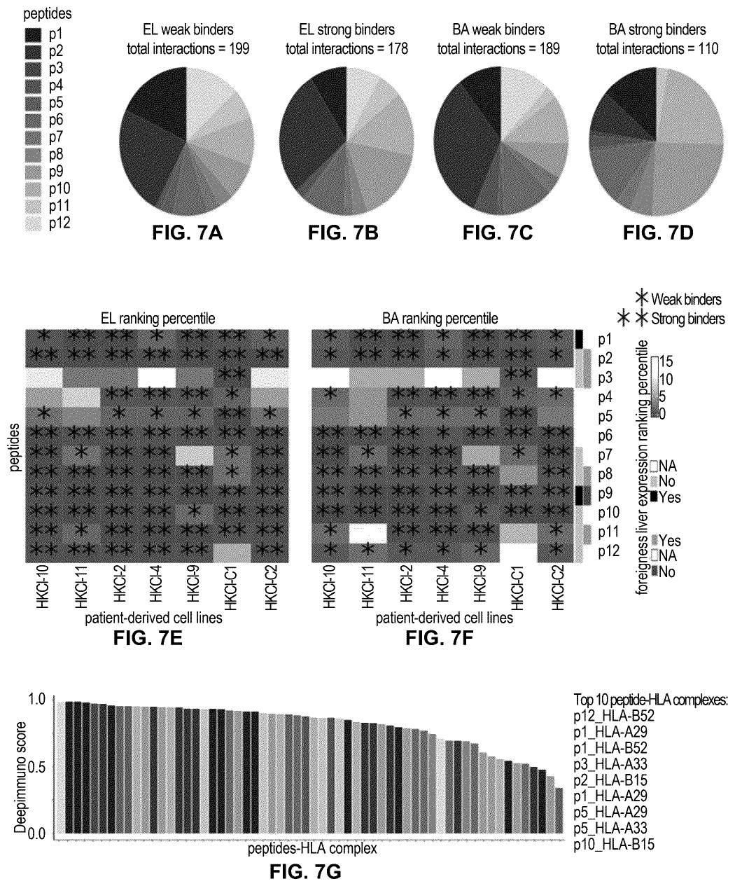

To assess the immunogenic potential of the non-canonical peptides, we focused on their NetMHCPan and IEAtlas results. p2 emerged as the most frequent significant HLA binder, indicating its potential for effective HLA presentation. p9 exhibited a notable enrichment for strong binding over weak binding, which may explain its strong immunogenicity demonstrated in the ELISpot analysis. Conversely, p3 was one of the least frequent HLA binders (FIG. 7a-d). Our analysis of p2 revealed strong binding interactions in all patients in EL prediction and four out of seven patients in BA prediction. Moreover, p2 exhibited predicted interactions with alleles for all seven cell lines, indicating its broad applicability as a potential therapeutic target. Notably. IEAtlas data on p2 expression showed non-tumor liver expression and homology to immunogenic antigens in Immune Epitope Database (foreignness) (FIG. 7e-f). We also observed similar patterns for p8 and p11, with p11 showing weaker HLA affinities across the patient samples.

The therapeutic value of tumor-specific antigens is contingent upon their ability to form TCR-peptide-MHC complexes. We assessed this criterion by employing DeepImmuno to query all NetMHCPan-predicted peptide-HLA complexes. Among the candidates exhibiting tumor specificity and foreignness, the p2-HLA-B15 complex emerged as one of the top-ranking complexes. Although p3 exhibited significant HLA interaction only with the HLA-A33 allele of the HKCI-C1 cell line, this interaction ranked among the top 10 peptide-HLA complexes with the highest TCR affinity. Furthermore, our analysis identified p10, a strong HLA binder for all patients except HKCI-9, as another peptide displaying tumor specificity and a high TCR affinity prediction score (FIG. 7g). Our results demonstrate that p2, p3, and p10 possess qualities that make them promising candidates for immunotherapeutic interventions.

In conclusion, our study identified and characterized a repertoire of twelve novel peptides derived from patient-specific tumor cell lines. Our findings contribute to elucidating HCC immunopeptidome and highlight potential avenues of therapeutic cancer vaccine for HCC.

### REFERENCES

[1] Rebecca L. Siegel, Kimberly D. Miller, Nikita Sandeep Wagle, and Ahmedin Jemal, Cancer statistics, 2023. CA: A Cancer Journal for Clinicians, 73(1):17-48, January 2023.
[2] Bruno Sangro, Carlos Gomez-Martin, Manuel de la Mata, Mercedes In\tilde{ }arrairaegui, Elena Garralda, Pilar Barrera, Jose Ignacio Riezu-Boj, Esther Larrea, Carlos Alfaro.,Pablo Sarobe, Juan Jos\acute{ }e Lasarte, Jose L P\acute{ }erez-Gracia, Ignacio Melero, and Jesu\acute{ }s Prieto. A clinical trial of CTLA-4 blockade with tremelimumab in patients with hepatocellular carcinoma and chronic hepatitis C. J. Hepatol., 59(1):81-88, July 2013.
[3] Anthony B El-Khoueiry. Bruno Sangro, Thomas Yau, Todd S Crocenzi, Masatoshi Kudo, Chiun Hsu, Tae-You Kim, Su-Pin Choo, J\ddot{ }org Trojan, Theodore H Welling, Rd, Tim Meyer, Yoon-Koo Kang, Winnie Yeo, Akhil Chopra, Jeffrey Anderson, Christine Dela Cruz, Lixin Lang, Jaclyn Neely, Hao Tang, Homa B Dastani, and Ignacio Melero. Nivolumab in patients with advanced hepatocellular carcinoma (CheckMate 040): an open-label, non-comparative, phase ½ dose escalation and expansion trial. Lancet, 389(10088):2492-2502, June 2017.
[4] Thomas Yau, Yoon-Koo Kang, Tae-You Kim, Anthony B El-Khoueiry, Armando Santoro, Bruno Sangro, Ignacio Melero, Masatoshi Kudo, Ming-Mo Hou, Ana Matilla, Francesco Tovoli, Jennifer J Knox, Aiwu Ruth He, Bassel F El-Rayes, Mirelis Acosta-Rivera, Ho-Yeong Lim, Jaclyn Neely, Yun Shen, Tami Wisniewski, Jeffrey Anderson. and Chiun Hsu. Efficacy and safety of nivolumab plus ipilimumab in patients with advanced hepatocellular carcinoma previously treated with sorafenib: The Check Mate 040) randomized clinical trial. JAMA Oncol., 6 (11):e204564, November 2020.
[5] Qin Li, Ziteng li, Bing Chen, Jingjing Zhao, Hw Yu, Jia Hu, Hongyan Lai, Hena Zhang, Yan Li, Zhiqiang Meng, Zhixiang Hu, and Shenglin Huang. Rna splicing junction landscape reveals abundant tumor-specific transcripts in human cancer. February 2024.
[6] Guangyuan Li, Shweta Mahajan, Siyuan Ma, Erin D. Jeffery, Xuan Zhang, Anukana Bhattacharjee, Meenakshi Venkatasubramanian, Matthew T. Weirauch, Emily R. Miraldi, H. Leighton Grimes, Glo-ria M. Sheynkman, Tamara Tilburgs, and Nathan Salomonis. Splicing neoantigen discovery with snaf reveals shared targets for cancer immunotherapy. Science Translational Medicine, 16(730), January 2024.
[7] Antonela Merlotti, Benjamin Sadacca, Yago A. Arribas, Mercia Ngoma, Marianne Burbage, Christel Goudot, Alexandre Houy, Ares Rocan\tilde{ }\acute{ }in-Arj\acute{ }o, Ana Lalanne, Agathe Seguin-Givelet, Marine Lefevre, Sandrine Heurtebise-Chr\acute{ }etien, Blandine Baudon. Giacomo Oliveira, Damarys Loew, Montserrat Car-rascal, Catherine J. Wu, Olivier Lantz, Marc-Henri Stern, Nicolas Girard, Joshua J. Waterfall, and Sebastian Amigorena. Noncanonical splicing junctions between exons and transposable elements repre-sent a source of immunogenic recurrent neo-antigens in patients with lung cancer. Science Immunology, 8(80)), February 2023.
[8] Nakul M. Shah, H. Josh Jang, Yonghao Liang, Ju Heon Maeng, Shin-Cheng Tzeng, Angela Wu, Noah L. Basri, Xuan Qu, Changxu Fan, Amy Li, Benjamin Katz, Daofeng Li, Xiaoyun Xing, Bradley S. Evans, and Ting Wang. Pan-cancer analysis identifies tumor-specific antigens derived from transposable ele-ments. Nature Genetics, 55(4):631-639, March 2023.
[9] Yu Kong, Christopher M. Rose, Ashley A. Cass, Alexander G. Williams, Martine Darwish, Steve Lianoglou, Peter M. Haverty, Ann-Jay Tong, Craig Blanchette, Matthew L. Albert, Ira Mellman, Richard Bourgon, John Greally, Suchit Jhunjhunwala, and Haiyin Chen-Harris. Transposable ele-ment expression in tumors is associated with 30) immune infiltration and increased antigenicity. Nature Communications, 10 (1), November 2019.
[10] Seunghyuk Choi and Eunok Paek. pxg: Comprehensive identification of noncanonical MHC-I-associated peptides from de novo peptide sequencing using RNA-Seq reads. Mol. Cell. Proteomics, (100743):100743, February 2024.
[11] Hui Chen, Feng Gao, Mian He, Xiao Fan Ding, Aikha M Wong, Siu Ching Sze, Allen C Yu, Tingting Sun, Anthony W-H Chan, Xin Wang, and Nathalie Wong. Long-read RNA sequencing identifies alternative splice variants in hepatocellular carcinoma and tumor-specific isoforms. Hepatology, 70(3):1011-1025, September 2019.
[12] Anthony W. Purcell, Sri H. Ramarathinam, and Nicola Ternette. Mass spectrometry-based identification of MHC bound peptides for immunopeptidomics. Nature Protocols, 14:1687-1707, 6 2019.
[13] Juan L. Trincado, Juan C. Entizne, Gerald Hysenaj, Babita Singh, Miha Skalic, David J. Elliott, and Eduardo Eyras. Suppa2: fast, accurate, and uncertainty-aware differential splicing analysis across multiple conditions. Genome Biology, 19(1), March 2018.
[14] Alexander Dobin, Carrie A Davis, Felix Schlesinger, Jorg Drenkow, Chris Zaleski, Sonali Jha, Philippe Batut, Mark Chaisson, and Thomas R Gingeras. STAR: ultrafast universal RNA-seq aligner. Bioinfor-matics, 29(1):15-21, January 2013.
[15] Mihaela Pertea, Geo M Pertea, Corina M Antonescu, Tsung-Cheng Chang, Joshua T Mendell, and Steven L Salzberg. Stringtie enables improved reconstruction of a transcriptome from rna-seq reads. Nature Biotechnology, 33(3):290-295, February 2015.
[16] Andrew J. Modzelewski, Wanqing Shao, Jingqi Chen, Angus Lee, Xin Qi, Mackenzie Noon, Kristy Tjokro, Gabriele Sales, Anne Biton, Aparna Anand, Terence P. Speed, Zhenyu Xuan, Ting Wang, Davide Risso, and Lin He. A mouse-specific retrotransposon drives a conserved cdk2ap1 isoform essential for development. Cell, 184(22):5541-5558.e22, October 2021.
[17] Runxuan Zhang, Richard Kuo, Max Coulter, Cristiane P. G. Calixto, Juan Carlos Entizne, Wenbin Guo, Yamile Marquez, Linda Milne, Stefan Riegler, Akihiro Matsui, Maho Tanaka, Sarah Harvey, Yubang Gao, Theresa Wießner-Kroh, Alejandro Paniagua, Martin Crespi, Katherine Denby, Asa ben Hur, Enamul Huq, Michael Jantsch, Artur Jarmolowski, Tino Koester, Sascha Laubinger, Qingshun Quinn Li, Lianfeng Gu, Motoaki Seki, Dorothee Staiger, Ramanjulu Sunkar, Zofia Szweykowska-Kulinska, Shih-Long Tu, Andreas Wachter, Robbie Waugh, Liming Xiong, Xiao-Ning Zhang, Ana Conesa, Anireddy S. N. Reddy, Andrea Barta, Maria Kalyna, and John W. S. Brown. A high-resolution single-molecule sequencing-based arabidopsis transcriptome using novel methods of iso-seq analysis. Genome Biology, 23(1), July 2022.
[18] Heng Li. Minimap2: pairwise alignment for nucleotide sequences. Bioinformatics, 34(18):3094-3100, May 2018.
[19] H ̊akon Tjeldnes, Kornel Labun, Yamila Torres Cleuren, Katarzyna Chyz\dot{ }yn\acute{ }ska, Micha-l S\acute{ }wirski, and Eivind Valen, Orfik: a comprehensive r toolkit for the analysis of translation. BMC Bioinformatics, 22(1), June 2021.
[20] Herv\acute{ }e Pag\grave{ }es, Paul Aboyoun, Robert Gentleman, and Sinisa DebRoy. Biostrings: Efficient manipulation of biological strings, 2024. R package version 2.70.2.
[21] Ju\ddot{ }rgen Cox and Matthias Mann. Maxquant enables high peptide identification rates. individual-ized p.p.b.-range mass accuracies and proteome-wide protein quantification. Nature Biotechnology, 26(12):1367-1372, November 2008.
[22] Chris Bielow, Guido Mastrobuoni, and Stefan Kempa. Proteomics quality control: Quality control software for maxquant results. Journal of Proteome Research, 15(3):777-787, December 2015.
[23] Joel N. H. Stern, Gur Yaari, Jason A. Vander Heiden, George Church, William F. Donahue, Rogier Q. Hintzen, Anita J. Huttner, Jon D. Laman. Rashed M. Nagra, Alyssa Nylander, David Pitt, Sriram Ramanan, Bilal A. Siddiqui, Francois Vigneault, Steven H. Kleinstein, David A. Hafler, and Kevin C. O'Connor. B cells populating the multiple sclerosis brain mature in the draining cervical lymph nodes. Science Translational Medicine, 6(248), August 2014.
[24] Namita T. Gupta, Jason A. Vander Heiden, Mohamed Uduman, Daniel Gadala-Maria, Gur Yaari, and Steven H. Kleinstein, Change-o: a toolkit for analyzing large-scale b cell immunoglobulin repertoire sequencing data. Bioinformatics, 31(20):3356-3358, June 2015.
[25] Kai Li, Marc Vaudel, Bing Zhang, Yan Ren, and Bo Wen. Pdv: an integrative proteomics data viewer. Bioinformatics, 35(7):1249-1251, August 2018.
[26] Birkir Reynisson, Bruno Alvarez, Sinu Paul, Bjoern Peters, and Morten Nielsen. Netmhcpan-4.1 and netmhciipan-4.0: improved predictions of mhc antigen presentation by concurrent motif deconvolution and integration of ms mhc eluted ligand data. Nucleic Acids Research, 48(W1):W449-W454, May 2020.
[27] Massimo Andreatta, Bruno Alvarez, and Morten Nielsen. Gibbscluster: unsupervised clustering and alignment of peptide sequences. Nucleic Acids Research, 45(W1):W458-W463, April 2017.
[28] Nicolas Rapin, Ilka Hoof, Ole Lund, and Morten Nielsen. Mhc motif viewer. Immunogenetics, 60(12):759-765, September 2008.
[29] Martin Christen Frolund Thomsen and Morten Nielsen. Seq2logo: a method for construction and visualization of amino acid binding motifs and sequence profiles including sequence weighting, pseudo counts and two-sided representation of amino acid enrichment and depletion. Nucleic Acids Research, 40(W1):W281-W287, May 2012.
[30] Guangyuan Li, Balaji Iver, V B Surya Prasath, Yizhao Ni, and Nathan Salomonis. Deepimmuno: deep learning-empowered prediction and generation of immunogenic peptides for t-cell immunity. Briefings in Bioinformatics, 22(6), May 2021.
[31] Rob Patro, Geet Duggal, Michael I Love, Rafael A Irizarry, and Carl Kingsford. Salmon provides fast and bias-aware quantification of transcript expression. Nat. Methods, 14(4):417-419, April 2017.
[32] John Lonsdale, Jeffrey Thomas, Mike Salvatore, Rebecca Phillips, Edmund Lo, Saboor Shad, Richard Hasz, Gary Walters, Fernando Garcia, Nancy Young, Barbara Foster, Mike Moser, Ellen Karasik, Bryan Gillard, Kimberley Ramsey, Susan Sullivan, Jason Bridge, Harold Magazine, John Syron, Johnelle Fleming, Laura Siminoff, Heather Traino, Maghboeba Mosavel, Laura Barker, Scott Jewell, Dan Rohrer, Dan Maxim, Dana Filkins, Philip Harbach, Eddie Cortadillo, Bree Berghuis, Lisa Turner, Eric Hudson, Kristin Feenstra, Leslie Sobin, James Robb, Phillip Branton, Greg Korzeniewski, Charles Shive, David Tabor, Liqun Qi, Kevin Groch, Sreenath Nampally, Steve Buia, Angela Zimmerman, Anna Smith, Robin Burges, Karna Robinson, Kim Valentino, Deborah Bradbury, Mark Cosentino, Norma Diaz-Mayoral, Mary Kennedy, Theresa Engel, Penelope Williams, Kenyon Erickson, Kristin Ardlie, Wendy Winckler, Gad Getz, David DeLuca, Daniel MacArthur, Manolis Kellis, Alexander Thomson, Taylor Young, Ellen Gelfand, Molly Donovan, Yan Meng, George Grant, Deborah Mash, Yvonne Marcus, Margaret Basile, Jun Liu, Jun Zhu, Zhidong Tu, Nancy J Cox, Dan L Nicolae, Eric R Gamazon, Hae Kyung Im, Anuar Konkashbaev, Jonathan Pritchard, Matthew Stevens, Timothee Flutre, Xiaoquan Wen, Emmanouil T Dermitzakis, Tuuli Lappalainen, Roderic Guigo, Jean Monlong, Michael Sammeth, Daphne Koller, Alexis Battle, Sara Mostafavi, Mark McCarthy, Manual Rivas, Julian Maller, Ivan Rusyn, Andrew Nobel, Fred Wright, Andrey Shabalin, Mike Feolo, Nataliya Sharopova, Anne Sturcke, Justin Paschal, James M Anderson, Elizabeth L Wilder, Leslie K Derr, Eric D Green, Jeffery P Struewing, Gary Temple, Simona Volpi, Joy T Bover, Elizabeth J Thomson, Mark S Guyer, Cathy Ng, Assya Abdallah, Deborah Colantuoni, Thomas R Insel, Susan E Koester, A Roger Little, Patrick K Bender, Thomas Lehner, Yin Yao, Carolyn C Compton, Jimmie B Vaught, Sherilyn Sawyer, Nicole C Lockhart, Joanne Demchok, and Helen F Moore. The genotype-tissue expression (gtex) project. Nature Genetics, 45(6):580-585, May 2013.
[33] Zuguang Gu, Roland Eils, and Matthias Schlesner. Complex heatmaps reveal patterns and correlations in multidimensional genomic data. Bioinformatics, 32(18):2847-2849, September 2016.
[34] James T Robinson, Helga Thorvaldsdottir, Wendy Winckler, Mitchell Guttman, Eric S Lander, Gad Getz, and Jill P Mesirov. Integrative genomics viewer. Nat. Biotechnol., 29(1):24-26. January 2011.
[35] Zefang Tang, Boxi Kang, Chenwei Li, Tianxiang Chen, and Zemin Zhang. Gepia2: an enhanced web server for large-scale expression profiling and interactive analysis. Nucleic Acids Research, 47(W1):W556-W560, May 2019.
[35] Zhanmin Liang, Haokai Ye, Jiongming Ma, Zhen Wei, Yue Wang, Yuxin Zhang, Daiyun Huang, Bowen Song, Jia Meng, Daniel J Rigden, and Kunqi Chen. m6a-atlas v2.0: updated resources for unravel-ing the n6-methyladenosine (m6a) epitranscriptome among multiple species. Nucleic Acids Research, 52(D1):D194-D202, August 2023.
[36] Shun Liu, Allen Zhu, Chuan He, and Mengjie Chen. Repic: a database for exploring the n6-methyladenosine methylome. Genome Biology, 21(1), April 2020.
[37] Jia-Jia Xuan, Wen-Ju Sun, Peng-Hui Lin, Ke-Ren Zhou, Shun Liu, Ling-Ling Zheng, Liang-Hu Qu, and Jian-Hua Yang. Rmbase v2.0: deciphering the map of rna modifications from epitranscriptome sequencing data. Nucleic Acids Research, 46(D1):D327-D334, October 2017.
[38] Qi Liu, Xin Peng, Mengyuan Shen, Qian Qian, Junlian Xing, Chen Li, and Richard I Gregory. Ribo-uorf: a comprehensive data resource of upstream open reading frames (uorfs) based on ribosome profiling. Nucleic Acids Research, 51(D1):D248-D261, November 2022.
[39] Yanyan Li, Honghong Zhou, Xiaomin Chen, Yu Zheng, Quan Kang, Di Hao, Lili Zhang, Tingrui Song, Huaxia Luo, Yajing Hao, Runsheng Chen, Peng Zhang, and Shunmin He. Smprot: A reliable repos-itory with comprehensive annotation of small proteins identified from ribosome profiling. Genomics, Proteomics amp; Bioinformatics, 19(4):602-610, August 2021.

All patents, patent applications, and other publications cited in this application, including published amino acid or polynucleotide sequences identified by GenBank Accession Numbers and the like, are incorporated by reference in the entirety for all purposes.

1. An isolate peptide consisting of the amino acid sequence of SEQ ID NO: 1, 2, 3, 4, 5, 6, 7, 8, 9, 10, 11, or 12, optionally with at least one heterologous amino acid sequence at the N-terminus and/or the C-terminus of the peptide.

2. The peptide of claim 1, which is no longer than 300 amino acids.

3. The peptide of claim 1, consisting of the amino acid sequence of SEQ ID NO: 1, 2, 3, 4, 5, 6, 7, 8, 9, 10, 11, or 12.

4. The peptide of claim 1, conjugated with a heterologous moiety.

5. The peptide of claim 3, wherein the heterologous moiety is a detectable moiety or a solid substrate.

6. A nucleic acid comprising a polynucleotide sequence encoding the peptide of claim 1.

7. The nucleic acid of claim 6, which is DNA or RNA.

8. The nucleic acid of claim 6, which is an expression cassette comprising a heterologous promoter sequence operably linked to the polynucleotide sequence.

9. A composition comprising (1) the peptide of claim 1 and (2) one or more pharmaceutically acceptable excipient.

10. The composition of claim 9, comprising an adjuvant.

11. The composition of claim 9, which is formulated for injection.

12. A method for diagnosing cancer in a patient, comprising detecting in a biological sample taken from the patient presence of at least one of the peptide of claim 1.

13. The method of claim 12, wherein the biological sample is a blood or tissue sample comprising lymphocytes.

14. The method of claim 12, wherein the biological sample is a peripheral blood mononucleated cell (PBMC) sample.

15. The method of claim 12, wherein the detecting comprises mass spectrometry.

16. The method of claim 12, wherein the presence of at least three peptides is detected, preferably the peptides having the amino acid sequences set forth in SEQ ID NOs: 2, 3, and 10.

17. The method of claim 12, wherein the cancer is liver cancer, preferably hepatocellular carcinoma (HCC).

18. A method for reducing risk of cancer or treating cancer in a patient in need thereof, comprising administering to the patient a composition comprising an effective amount of the peptide of claim 1.

19. The method of claim 18, wherein the patient is at risk of developing liver cancer or has received a diagnosis of liver cancer.

20. The method of claim 18, wherein the cancer is hepatocellular carcinoma (HCC).

21. The method of claim 18, wherein the administering comprises injecting the composition to the patient.

22. A kit for inhibiting cancer cell proliferation, comprising a first container containing the composition of claim 9 and a second container containing an anti-cancer therapeutic agent.

23. The kit of claim 22, wherein the composition comprises the peptides having the amino acid sequences set forth in SEQ ID NOs: 2, 3, and 10.

24. The kit of claim 22, wherein the cancer is hepatocellular carcinoma (HCC).

25. The kit of claim 22, further comprising user instructions.
